(19) United States
(12) Patent Application Publication
(10) Pub. No.: US 2022/0195518 A1
(43) Pub. Date: Jun. 23, 2022

(54) METHODS AND COMPOSITIONS FOR NUCLEIC ACID SEQUENCING

(71) Applicant: Illumina Cambridge Limited, Cambridge (GB)

(72) Inventors: Xiaolin WU, Cambridge (GB); Carole ANASTASI, Cambridge (GB); Geraint EVANS, Cambridge (GB); Xiaohai LiU, Cambridge (GB)

(21) Appl. No.: 17/555,945

(22) Filed: Dec. 20, 2021

Related U.S. Application Data
(60) Provisional application No. 63/129,137, filed on Dec. 22, 2020.

(51) Int. Cl. C12Q 1/6874

(52) U.S. Cl. CPC C12Q 1/6874

(57) ABSTRACT

Embodiments of the present disclosure relate to methods, kits and compositions for two-channel nuclei acid sequencing using blue and violet light excitation (e.g., lasers at 450-460 nm and 400-405 nm respectively). In particular, the nucleotides may be directly labeled with a blue dye, a violet dye, or both a blue dye and a violet dye. Alternatively, one or more nucleotides for incorporation may be unlabeled and affinity reagents containing a blue dye, a violet dye, or both a blue dye and a violet dye may be used to bind specifically to each type of nucleotides incorporated.

### INCORPORATION BY REFERENCE TO ANY PRIORITY APPLICATION

The present application claims the benefit of priority to U.S. Ser. No. 63/129,137, filed Dec. 22, 2020, which is incorporated by reference in its entirety.

### FIELD

The present disclosure generally relates to methods, systems, kits and compositions for nuclei acid sequencing applications.

### BACKGROUND

For DNA sequencing, it is desirable to employ multiple spectrally distinguishable fluorescent labels to achieve independent detection of a plurality of spatially overlapping analytes. In such multiplex methods, the number of reaction vessels may be reduced, simplifying experimental protocols and facilitating the production of application-specific reagent kits. In multi-color automated DNA sequencing systems for example, multiplex fluorescent detection allows for the analysis of multiple nucleotide bases in a single electrophoresis lane, thereby increasing throughput over single-color methods, and reducing uncertainties associated with inter-lane electrophoretic mobility variations.

However, multiplex fluorescent detection can be problematic and there are a number of important factors that constrain selection of appropriate fluorescent labels. First, it may be difficult to find dye compounds with substantially resolved absorption and emission spectra in a given application. In addition, when several fluorescent dyes are used together, generating fluorescence signals in distinguishable spectral regions by simultaneous excitation may be complicated because absorption bands of the dyes are usually widely separated, so it is difficult to achieve comparable fluorescence excitation efficiencies even for two dyes. Many excitation methods use high power light sources like lasers and therefore the dye must have sufficient photo-stability to withstand such excitation. A final consideration of particular importance to molecular biology methods is the extent to which the fluorescent dyes must be compatible with reagent chemistries such as, for example, DNA synthesis solvents and reagents, buffers, polymerase enzymes, and ligase enzymes.

Fluorescent dye molecules with improved fluorescence properties such as suitable fluorescence intensity, shape, and wavelength maximum of fluorescence band can improve the speed and accuracy of nucleic acid sequencing. Strong fluorescence signals are especially important when measurements are made in water-based biological buffers and at higher temperatures as the fluorescence intensities of most organic dyes are significantly lower under such conditions. Moreover, the nature of the base to which a dye is attached also affects the fluorescence maximum, fluorescence intensity, and others spectral dye properties. The sequence-specific interactions between the nucleobases and the fluorescent dyes can be tailored by specific design of the fluorescent dyes. Optimization of the structure of the fluorescent dyes can improve the efficiency of nucleotide incorporation, reduce the level of sequencing errors, and decrease the usage of reagents in, and therefore the costs of, nucleic acid sequencing.

Some optical and technical developments have already led to greatly improved image quality but were ultimately limited by poor optical resolution. Optical resolution is dictated by Abbe's law. Generally, optical resolution of light microscopy is limited to objects spaced at approximately half of the wavelength of the light used. In practical terms, only objects that are laying quite far apart (at least 200 to 350 nm) could be resolved by light microscopy. One way to improve image resolution and increase the number of resolvable objects per unit of surface area is to use excitation light of a shorter wavelength. For example, if light wavelength is shortened by Δλ˜100 nm with the same optics, resolution will be better (about Δ 50 nm/(about 15%)), less-distorted images will be recorded, and the density of objects on the recognizable area will be increased about 35%.

However, extensive laser irradiation, especially shorter wavelength in the blue or violet regions, may bleach fluorescent dyes and damage nucleotide samples in solution/on flow-cell surface or those to which the fluorescent dyes are conjugated. Such expose to light may also cause DNA sample damage. The type and extent of photo-bleaching and photo-damages may vary depending on, for example, compounds chemical structure and some their physical-chemical properties like redox potential, excitation spectra of particular bio-label, intensity of particular light source irradiation, and time of exposure in particular measurement. Since lower wavelength light sources are delivering higher energy photons, violet LED/laser having shorter wavelength are more likely to cause photo-bleaching of dyes and DNA damage. There are a number of chemical pathways by which nucleic acid damage can occur during irradiation in fluorescence detection. For example, it has been indicated that exposure to ultraviolet (UV) radiation can cause DNA damage via the direct photochemical [2+2] photocycloaddition reaction of thymine or cytosine to provide cyclobutane containing fused pyrimidine dimers, such as TT, TC, and CC. Such direct photocycloaddition reactions can occur in the UV B and UV C regions which extend from about 100 nm to about 315 nm. In the UV A region through a portion of the visible region, spanning from about 315 nm to about 500 nm, a complex mixture of indirect mechanisms can also cause DNA damage through photosensitization of other components. Such indirect mechanisms can result oxidative DNA modification via interaction with different light induced reactive species, for example, Reactive Oxygen Species (ROS) such as singlet oxygen, superoxide anion, and hydroxyl radical.

In order to increase sequencing efficiency and decrease cost per genome, it is essential to increase the pitch density such that more clusters can be packed in the same required surface area while maintaining good optical resolution, which requires the use of lights with shorter wavelengths (such as violet and blue lasers). There remains a challenge to select the appropriate set of dyes in a very crowded region of wavelength (blue to violet region) for nucleic acid sequencing applications and mitigate DNA damage caused by shorter wavelength excitation.

### SUMMARY

The present disclosure relates to methods, kits and compositions for two-channel nuclei acid sequencing applications using blue and violet light excitation (e.g., lasers at 450-460 nm and 400-405 nm).

Some aspects of the present disclosure relate to a method for determining the sequence of a target polynucleotide, comprising:

(a) contacting a primer polynucleotide with a mixture comprising one or more of a first type of nucleotide, a second type of nucleotide, a third type of nucleotide, and a fourth type of nucleotide, wherein the primer polynucleotide is complementary to at least a portion of the target polynucleotide;

(b) incorporating one type of nucleotide from the mixture to the primer polynucleotide to produce an extended primer polynucleotide;

(c) performing a first imaging event using a first excitation light source and collecting a first emission signal from the extended primer polynucleotide with a first emission filer; and

(d) performing a second imaging event using a second excitation light source and collecting a second emission signal from the extended primer polynucleotide with a second emission filter;

wherein one of the first excitation light source and the second excitation light source has a wavelength of about 350 nm to about 410 nm, and the other one of the first excitation light source and the second excitation light source has a wavelength of about 450 nm to about 460 nm; and

wherein one of the first emission filter and the second emission filter has a detection wavelength of about 415 nm to about 450 nm, and the other one of the first emission filter and the second emission filter has a detection wavelength of about 480 nm to about 525 nm. In some embodiments, each of the first type, the second type and the third type of nucleotide is labeled with a detectable label. In other embodiments, one or more of the first type, the second type and the third type of nucleotide is unlabeled and the method utilizes a second labeling step involving the use of one or more affinity reagents that binds specifically to an unlabeled nucleotide that is incorporated into the primer polynucleotide/target polynucleotide complex. In further embodiments, the fourth type of nucleotide is unlabeled and does not emit any signal during the first imaging event and the second imaging event.

Some aspects of the present disclosure relate to a kit for sequencing application, comprising:

a first type of nucleotide labeled with a first detectable label;

a second type of nucleotide labeled with a second detectable label;

a third type of nucleotide labeled with the first detectable label; and

a third type of nucleotide labeled with the second detectable label;

wherein the first detectable label and the second detectable label are spectrally distinguishable from one another, the first detectable label is excitable by a first light source and detectable by a first emission filter, and the second detectable label is excitable by a second light source and detectable by a second emission filter;

wherein one of the first excitation light source and the second excitation light source has a wavelength of about 350 nm to about 410 nm, and the other one of the first excitation light source and the second excitation light source has a wavelength of about 450 nm to about 460 nm; and

wherein one of the first emission filter and the second emission filter has a detection wavelength of about 415 nm to about 450 nm, and the other one of the first emission filter and the second emission filter has a detection wavelength of about 480 nm to about 525 nm.

Some aspects of the present disclosure relate to a kit for sequencing application, comprising:

a first type of nucleotide labeled with a first detectable label;

a second type of nucleotide labeled with a second detectable label;

a third type of nucleotide labeled with a third detectable label; and

a third type of nucleotide labeled with a fourth detectable label;

wherein the first detectable label and the second detectable label are spectrally distinguishable from one another, the first detectable label is excitable by a first light source and detectable by a first emission filter, and the second detectable label is excitable by a second light source and detectable by a second emission filter;

wherein the third detectable label and the fourth detectable label are spectrally distinguishable from one another, the third detectable label is excitable by the first light source and detectable by the first emission filter, and the fourth detectable label is excitable by the second light source and detectable by the second emission filter;

wherein one of the first excitation light source and the second excitation light source has a wavelength of about 350 nm to about 410 nm, and the other one of the first excitation light source and the second excitation light source has a wavelength of about 450 nm to about 460 nm; and

wherein one of the first emission filter and the second emission filter has a detection wavelength of about 415 nm to about 450 nm, and the other one of the first emission filter and the second emission filter has a detection wavelength of about 480 nm to about 525 nm.

Some other aspects of the present disclosure relate to a kit for sequencing application, comprising:

a first type of unlabeled nucleotide;

a second type of unlabeled nucleotide;

a third type of unlabeled nucleotide; and

a set of affinity reagents comprising:
a first affinity reagent that binds specifically to the first type of unlabeled nucleotide; and
a second affinity reagent that binds specifically to the second type of unlabeled nucleotide;
wherein the first affinity reagent comprises one or more first detectable labels that are excitable by a first excitation light source and detectable by a first emission filter, the second affinity reagent comprises one or more second detectable labels that are excitable by a second excitation light source and detectable by a second emission filter, and wherein the first detectable label is spectrally distinguishable from the second detectable label;

wherein one of the first excitation light source and the second excitation light source has a wavelength of about 350 nm to about 410 nm, and the other one of the first excitation light source and the second excitation light source has a wavelength of about 450 nm to about 460 nm; and

wherein one of the first emission filter and the second emission filter has a detection wavelength of about 415 nm to about 450 nm, and the other one of the first emission filter and the second emission filter has a detection wavelength of about 480 nm to about 525 nm. In some embodiments, both the first affinity reagent and the second affinity reagent bind specifically to the third type of unlabeled nucleotide. In other embodiments, the set of affinity reagents further comprises a third affinity reagent that binds specifically to the third type of nucleotide, and wherein the third affinity reagent comprises one or more third detectable labels that are excitable by the first excitation light source and detectable by the first emission filter, and one or more fourth detectable labels that are excitable by the second excitation light source and detectable by the second emission filter.

Some other aspects of the present disclosure relate to a kit for sequencing application, comprising:

a first type of nucleotide either unlabeled or labeled with a first detectable label;

a second type of nucleotide either unlabeled or labeled with a second detectable label, wherein one of the first type of nucleotide and the second type of nucleotide is unlabeled;

a third type of unlabeled nucleotide, and a third type of nucleotide labeled with the same detectable label as either the first or the second type of nucleotide, wherein the first detectable label and the second detectable label are spectrally distinguishable from one another, the first detectable label is excitable by a first light source and detectable by a first emission filter, and the second detectable label is excitable by a second light source and detectable by a second emission filter; and

an affinity reagent comprising either a first affinity reagent that binds specifically to the third type of unlabeled nucleotide and the first type of nucleotide if the first type of nucleotide is unlabeled, or a second affinity reagent that binds specifically to the third type of unlabeled nucleotide and the second type of nucleotide if the second type of nucleotide is unlabeled, wherein the first affinity reagent comprises one or more first detectable labels and the second affinity reagent comprises one or more second detectable labels;

wherein one of the first excitation light source and the second excitation light source has a wavelength of about 350 nm to about 410 nm, and the other one of the first excitation light source and the second excitation light source has a wavelength of about 450 nm to about 460 nm; and

wherein one of the first emission filter and the second emission filter has a detection wavelength of about 415 nm to about 450 nm, and the other one of the first emission filter and the second emission filter has a detection wavelength of about 480 nm to about 525 nm.

### BRIEF DESCRIPTION OF THE DRAWINGS

FIG. 1 is a line chart illustrating the DNA photodamage caused by violet light exposure as a function of time

FIG. 2 is a scatter plot obtained with a secondary labeling sequencing by synthesis method described in Example 2.

### DETAILED DESCRIPTION

The present disclosure relates to methods, systems, kits and compositions for nuclei acid sequencing applications, in particular sequencing by synthesis, using blue and violet light excitation (e.g., lasers at 450-460 nm and 400-405 nm) and two-channel detection using filter bands at about 415-450 nm and about 480-525 nm. The methods, kits and compositions described herein utilize a dye set including blue and violet dyes (i.e., dyes with absorption maximum at the blue light and violent light regions). The methods further utilize affinity reagents to reduce the DNA damage and photo-bleaching caused by blue and violet excitations. The sequencing methods described herein with shorter wavelength light sources can increase pitch or cluster density on the patterned arrays or flow cells compared to the current two-channel sequencing used on Illumina's MiniSeq®, NextSeq®, and NovaSeq® systems, which use the red/green light source excitation or green/blue light source excitation. The term “pitch” as used herein, refers to the distance between two nanopatterns on a patterned solid support (e.g., the distance between two nanowells on a patterned flowcell). Detailed description for the Illumina two-channel sequencing using red/green light source excitation is disclosed in U.S. Patent Publication No. 2013/0079232, which is incorporated herein by reference in its entirety. For example, in a system using green/red or blue/green excitation, the optical resolution is limited by the red fluorescent dye or green fluorescent dye emission respectively (e.g., at about 715 nm for green/red system and about 590 nm for the blue/green system). By using violet/blue light excitation, the optical resolution is limited by blue dye emission (i.e., 480-525 nm). As such, the methods and systems of the present disclosure may offer up to 50% increase in pitch density.

### Definitions

Unless defined otherwise, all technical and scientific terms used herein have the same meaning as is commonly understood by one of ordinary skill in the art. The use of the term “including” as well as other forms, such as “include”, “includes,” and “included,” is not limiting. The use of the term “having” as well as other forms, such as “have”, “has,” and “had,” is not limiting. As used in this specification, whether in a transitional phrase or in the body of the claim, the terms “comprise(s)” and “comprising” are to be interpreted as having an open-ended meaning. That is, the above terms are to be interpreted synonymously with the phrases “having at least” or “including at least.” For example, when used in the context of a process, the term “comprising” means that the process includes at least the recited steps but may include additional steps. When used in the context of a compound, composition, or device, the term “comprising” means that the compound, composition, or device includes at least the recited features or components, but may also include additional features or components.

As used herein, common organic abbreviations are defined as follows:
° C. Temperature in degrees Centigrade
dATP Deoxyadenosine triphosphate
dCTP Deoxycytidine triphosphate
dGTP Deoxyguanosine triphosphate
dTTP Deoxythymidine triphosphate
ddNTP Dideoxynucleotide triphosphate
ffA Fully functionalized A nucleotide
ffC Fully functionalized C nucleotide
ffG Fully functionalized G nucleotide
ffN Fully functionalized nucleotide
ffT Fully functionalized T nucleotide
LED Light emitting diode
SBS Sequencing by synthesis

As used herein, the term “array” refers to a population of different probe molecules that are attached to one or more substrates such that the different probe molecules can be differentiated from each other according to relative location. An array can include different probe molecules that are each located at a different addressable location on a substrate. Alternatively, or additionally, an array can include separate substrates each bearing a different probe molecule, wherein the different probe molecules can be identified according to the locations of the substrates on a surface to which the substrates are attached or according to the locations of the substrates in a liquid. Exemplary arrays in which separate substrates are located on a surface include, without limitation, those including beads in wells as described, for example, in U.S. Pat. No. 6,355,431 B1, US 2002/0102578 and PCT Publication No. WO 00/63437. Exemplary formats that can be used in the invention to distinguish beads in a liquid array, for example, using a microfluidic device, such as a fluorescent activated cell sorter (FACS), are described, for example, in U.S. Pat. No. 6,524,793. Further examples of arrays that can be used in the invention include, without limitation, those described in U.S. Pat. Nos. 5,429,807; 5,436,327; 5,561,071; 5,583,211; 5,658,734; 5,837,858; 5,874,219; 5,919,523; 6,136,269; 6,287,768; 6,287,776; 6,288,220; 6,297,006; 6,291,193; 6,346,413; 6,416,949; 6,482,591; 6,514,751 and 6,610,482; and WO 93/17126; WO 95/11995; WO 95/35505; EP 0 742 287; and EP 0 799 897.

As used herein, the term “covalently attached” or “covalently bonded” refers to the forming of a chemical bonding that is characterized by the sharing of pairs of electrons between atoms. For example, a covalently attached polymer coating refers to a polymer coating that forms chemical bonds with a functionalized surface of a substrate, as compared to attachment to the surface via other means, for example, adhesion or electrostatic interaction. It will be appreciated that polymers that are attached covalently to a surface can also be bonded via means in addition to covalent attachment.

In each instance where a single mesomeric form of a compound described herein is shown, the alternative mesomeric forms are equally contemplated.

As used herein, a “nucleotide” includes a nitrogen containing heterocyclic base, a sugar, and one or more phosphate groups. They are monomeric units of a nucleic acid sequence. In RNA, the sugar is a ribose, and in DNA a deoxyribose, i.e. a sugar lacking a hydroxyl group that is present in ribose. The nitrogen containing heterocyclic base can be purine, deazapurine, or pyrimidine base. Purine bases include adenine (A) and guanine (G), and modified derivatives or analogs thereof, such as 7-deaza adenine or 7-deaza guanine. Pyrimidine bases include cytosine (C), thymine (T), and uracil (U), and modified derivatives or analogs thereof. The C-1 atom of deoxyribose is bonded to N-1 of a pyrimidine or N-9 of a purine. In some instance, the term “nucleotide” may also encompass a nucleotide conjugate, which is a nucleotide labeled with a fluorescent moiety, optionally through a cleavage linker as described herein.

As used herein, an “unlabeled nucleotide” refers to a nucleotide that does not include a fluorescent moiety. In some instances, an unlabeled nucleotide may comprise a cleavable linker and/or a functional moiety (e.g., a hapten) that allows it to bind to an affinity reagent described herein. In other instances, an unlabeled nucleotide does not have a cleavable linker or a functional moiety that allows it to bind to an affinity reagent described herein.

As used herein, a “nucleoside” is structurally similar to a nucleotide but is missing the phosphate moieties. An example of a nucleoside analogue would be one in which the label is linked to the base and there is no phosphate group attached to the sugar molecule. The term “nucleoside” is used herein in its ordinary sense as understood by those skilled in the art. Examples include, but are not limited to, a ribonucleoside comprising a ribose moiety and a deoxyribonucleoside comprising a deoxyribose moiety. A modified pentose moiety is a pentose moiety in which an oxygen atom has been replaced with a carbon and/or a carbon has been replaced with a sulfur or an oxygen atom. A “nucleoside” is a monomer that can have a substituted base and/or sugar moiety. Additionally, a nucleoside can be incorporated into larger DNA and/or RNA polymers and oligomers.

The term “purine base” is used herein in its ordinary sense as understood by those skilled in the art and includes its tautomers. Similarly, the term “pyrimidine base” is used herein in its ordinary sense as understood by those skilled in the art and includes its tautomers. A non-limiting list of optionally substituted purine-bases includes purine, adenine, guanine, deazapurine, 7-deaza adenine, 7-deaza guanine, hypoxanthine, xanthine, alloxanthine, 7-alkylguanine (e.g. 7-methylguanine), theobromine, caffeine, uric acid and isoguanine. Examples of pyrimidine bases include, but are not limited to, cytosine, thymine, uracil, 5,6-dihydrouracil and 5-alkylcytosine (e.g., 5-methylcytosine).

As used herein, when an oligonucleotide or polynucleotide is described as “comprising” a nucleoside or nucleotide described herein, it means that the nucleoside or nucleotide described herein forms a covalent bond with the oligonucleotide or polynucleotide. Similarly, when a nucleoside or nucleotide is described as part of an oligonucleotide or polynucleotide, such as “incorporated into” an oligonucleotide or polynucleotide, it means that the nucleoside or nucleotide described herein forms a covalent bond with the oligonucleotide or polynucleotide. In some such embodiments, the covalent bond is formed between a 3′ hydroxy group of the oligonucleotide or polynucleotide with the 5′ phosphate group of a nucleotide described herein as a phosphodiester bond between the 3′ carbon atom of the oligonucleotide or polynucleotide and the 5′ carbon atom of the nucleotide.

As used herein, the term “cleavable linker” is not meant to imply that the whole linker is required to be removed. The cleavage site can be located at a position on the linker that ensures that part of the linker remains attached to the detectable label and/or nucleoside or nucleotide moiety after cleavage.

As used herein, “derivative” or “analog” means a synthetic nucleotide or nucleoside derivative having modified base moieties and/or modified sugar moieties. Such derivatives and analogs are discussed in, e.g., Scheit, Nucleotide Analogs (John Wiley & Son, 1980) and Uhlman et al., Chemical Reviews 90:543-584, 1990. Nucleotide analogs can also comprise modified phosphodiester linkages, including phosphorothioate, phosphorodithioate, alkyl-phosphonate, phosphoranilidate and phosphoramidate linkages. “Derivative”, “analog” and “modified” as used herein, may be used interchangeably, and are encompassed by the terms “nucleotide” and “nucleoside” defined herein.

As used herein, the term “phosphate” is used in its ordinary sense as understood by those skilled in the art, and includes its protonated forms

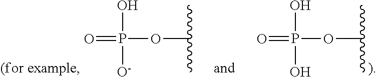 [SMILES: *OP(=O)([O-])O ; *OP(=O)(O)O]

As used herein, the terms “monophosphate,” “diphosphate,” and “triphosphate” are used in their ordinary sense as understood by those skilled in the art and include protonated forms.

As understood by one of ordinary skill in the art, a compound such as a nucleotide described herein may exist in ionized form, e.g., containing a —CO₂⁻, —SO₃⁻ or —O⁻. If a compound contains a positively or negatively charged substituent group, it may also contain a negatively or positively charged counterion such that the compound as a whole is neutral. In other aspects, the compound may exist in a salt form, where the counterion is provided by a conjugate acid or base.

As used herein, the term “phasing” refers to a phenomenon in SBS that is caused by incomplete removal of the 3′ terminators and fluorophores, and/or failure to complete the incorporation of a portion of DNA strands within clusters by polymerases at a given sequencing cycle. Prephasing is caused by the incorporation of nucleotides without effective 3′ terminators, wherein the incorporation event goes 1 cycle ahead due to a termination failure. Phasing and prephasing cause the measured signal intensities for a specific cycle to consist of the signal from the current cycle as well as noise from the preceding and following cycles. As the number of cycles increases, the fraction of sequences per cluster affected by phasing and prephasing increases, hampering the identification of the correct base. Prephasing can be caused by the presence of a trace amount of unprotected or unblocked 3′-OH nucleotides during sequencing by synthesis (SBS). The unprotected 3′-OH nucleotides could be generated during the manufacturing processes or possibly during the storage and reagent handling processes.

As used herein, the term “spectrally distinguishable fluorescent dyes” refers to fluorescent dyes that emit fluorescent energy at wavelengths that can be distinguished by fluorescent detection equipment when two or more such dyes are present in one sample.

### Blue/Violet Two-Channel Sequencing Methods

Some aspects of the present disclosure relate to a method for determining the sequence of a target polynucleotide (e.g., a single stranded target polynucleotide), comprising:

(a) contacting a primer polynucleotide/target polynucleotide complex with a mixture comprising one or more of a first type of nucleotide, a second type of nucleotide, a third type of nucleotide, and a fourth type of nucleotide, wherein the primer polynucleotide is complementary to at least a portion of the single stranded target polynucleotide;

(b) incorporating one type of nucleotide from the mixture to the primer polynucleotide to produce an extended primer polynucleotide (i.e., an extended primer polynucleotide/target polynucleotide complex);

(c) performing a first imaging event using a first excitation light source and collecting a first emission signal from the extended primer polynucleotide/target polynucleotide complex with a first emission filer; and

(d) performing a second imaging event using a second excitation light source and collecting a second emission signal from the extended primer polynucleotide/target polynucleotide complex with a second emission filter;

wherein one of the first excitation light source and the second excitation light source has a wavelength of about 350 nm to about 410 nm, and the other one of the first excitation light source and the second excitation light source has a wavelength of about 450 nm to about 460 nm; and

wherein one of the first emission filter and the second emission filter has a detection wavelength of about 415 nm to about 450 nm, and the other one of the first emission filter and the second emission filter has a detection wavelength of about 480 nm to about 525 nm.

In some embodiments of the method described herein, the first excitation light source has a wavelength of about 350 nm to about 410 nm (e.g., about 405 nm), and the first emission filter has a detection wavelength of about 415 nm to about 450 nm. The second excitation light source has a wavelength of about 450 nm to about 460 nm (e.g., about 460 nm), and the second emission filter has a detection wavelength of about 480 nm to about 525 nm. In some other embodiments, the first excitation light source has a wavelength of about 450 nm to about 460 nm (e.g., about 460 nm), and the first emission filter has a detection wavelength of about 480 nm to about 525 nm. The second excitation light source has a wavelength of about 350 nm to about 410 nm (e.g., about 405 nm), and the second emission filter has a detection wavelength of about 415 nm to about 450 nm.

### Labeled Nucleotide in Incorporation Mixture

In some embodiments of the method described herein, each type of nucleotides in the incorporation mixture is labeled. In some such embodiments, the first type of nucleotide is labeled with a first detectable label that is excitable by the first excitation light source and detectable by the first emission filter. In some further embodiments, the second type of nucleotide is labeled with a second detectable label that is excitable by the second excitation light source and detectable by the second emission filter, and wherein the second type of detectable label is spectrally distinguishable from the first type of detectable label. In some further embodiments, the third type of nucleotide is labeled both with a first detectable label and a second detectable label, and the third type of nucleotide is excitable by both the first excitation light source and the second excitation light source. In some other embodiments, the third type of nucleotide comprises a mixture of a third type of nucleotide labeled with a third label and a third type of nucleotide labeled with a fourth label, wherein the third label is excitable by the first excitation light source and detectable by the first emission filter, and wherein the fourth label is excitable by the second excitation light source and detectable by the second emission filter. In further embodiments, the fourth type of nucleotide is not unlabeled, or is labeled with a fluorescent moiety that does not have any emission under either the first or the second imaging event. In some instances, the fourth type of nucleotide contains a G base (e.g., dGTP).

When a type of nucleotide is described as labeled with two different labels, it includes the following two scenarios. In the first scenario, the nucleotide is a mixture of the nucleotide labeled with a first label and the same type of nucleotide labeled with a second label. In the second scenario, the nucleotide has both the first label and the second label covalently attached thereto (i.e., two labels on the same molecule). In addition, the type of nucleotide described as labeled with a first and a second labels may also include one or more additional detectable labels that are different from the first label and the second label.

As a first example, the first type of nucleotide may be labeled with a first dye that is excitable by a blue light source at about 450-460 nm (i.e., the first dye is a blue dye) and has an emission wavelength in the range of 480-525 nm. The second type of nucleotide may be labeled with a second dye that is excitation by a violet light source at about 400-405 nm (i.e., the second dye is a violet dye) and has an emission wavelength in the range of 415-450 nm. The third type of nucleotide may be a mixture of the third nucleotide labeled with the first dye and the third nucleotide labeled with the second dye. The fourth type of nucleotide is unlabeled. The first imaging event uses a blue light source having a wavelength of about 450-460 nm, and both the first type and the third type of nucleotides will emit a signal that can be detected or collected by an emission filter having a filter band that encompasses 480-525 nm. The second imaging event uses a violet light source having a wavelength of about 400-405 nm, and both the second type and the third type of nucleotides will emit a signal that can be detected or collected by an emission filter having a filter band that encompasses 415-450 nm. Since the fourth type of nucleotide is not labeled, no signal will be detected under either the first or the second imaging event. Based on the signal detection pattern described herein, the identity of the incorporated nucleotide in the extended primer polynucleotide/target polynucleotide complex may be determined. In a further embodiment, the incorporation mixture comprises the following: a dATP labeled with a blue dye A, a dTTP labeled with a violet dye B, a dCTP labeled with the blue dye A, a dCTP labeled with the violet dye B, and an unlabeled dGTP (dark G). In one embodiment, the dATP labeled with a blue dye may have the following structure:

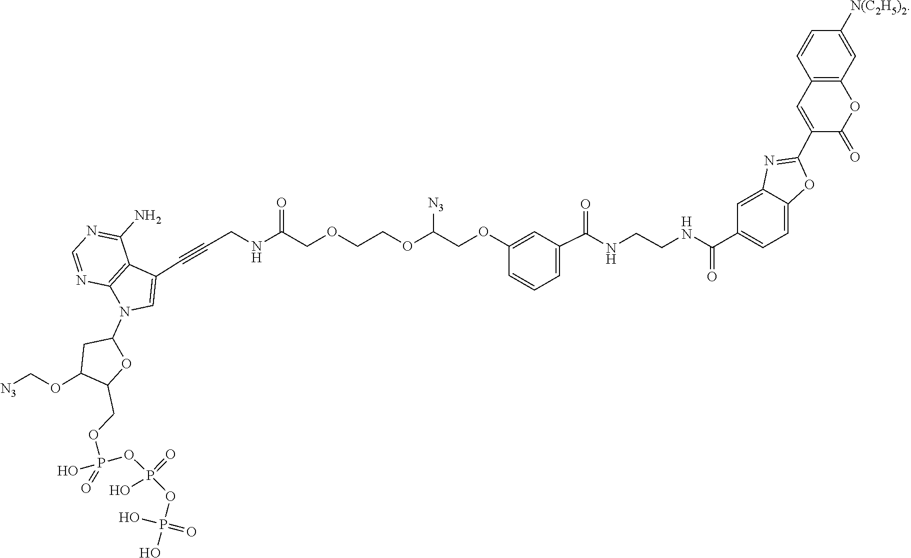

Such A nucleotide dye conjugate is also referred to as fully functionalized A nucleotide (ffA).

As a second example, the first type of labeled nucleotide, the second type of labeled nucleotide and the fourth type of unlabeled nucleotide are the same as those described in the first example. The third type of nucleotide may be labeled with both a third dye and a fourth dye (e.g., a mixture of a third type nucleotide labeled with the third dye and a third type of nucleotide labeled with the fourth dye. The third dye has similar fluorescent profile as the first dye (i.e., absorption and emission spectra) but may be different in terms of emission intensity. The fourth dye has similar fluorescent profile as the second dye (i.e., absorption and emission spectra) but may be different in terms of emission intensity. In a further embodiment, the incorporation mixture comprises the following: a dATP labeled with a blue dye A, a dTTP labeled with a violet dye B, a dCTP labeled with the blue dye C, a dCTP labeled with the violet dye D, and an unlabeled dGTP (dark G).

Violet Dyes

Fluorescent dyes that are excitable by a violet light source having a wavelength of about 350-405 nm may be used as the first or the second detectable label described herein. In further embodiments, particularly useful violet dyes may have emission spectra in the range of 410-460 nm or 415-450 nm. Non-limiting examples of the violet dyes include:

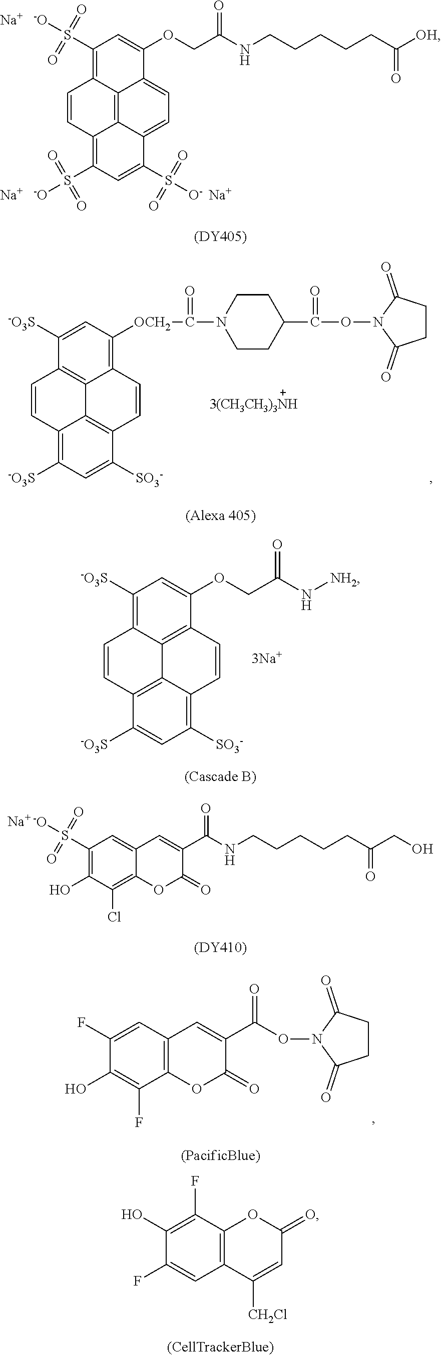

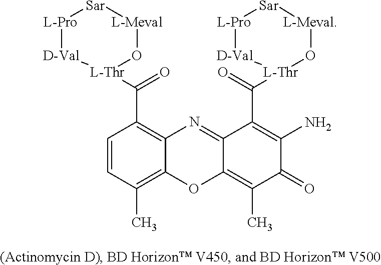

Blue Dyes

Fluorescent dyes that are excitable by a blue light source having a wavelength of about 450-460 nm may be used as the first or the second detectable label described herein. In further embodiments, particularly useful blue dyes may have emission spectra in the range of 475-530 nm or 480-525 nm. Non-limiting examples of the blue dyes include coumarin dyes disclosed in U.S. Publication Nos. 2018/0094140 A1, 2018/0201981 A1, 2020/0277529 A1 and 2020/0277670 A1, which are incorporated herein by reference.

In some embodiments, non-limiting exemplary blue dyes include the following:

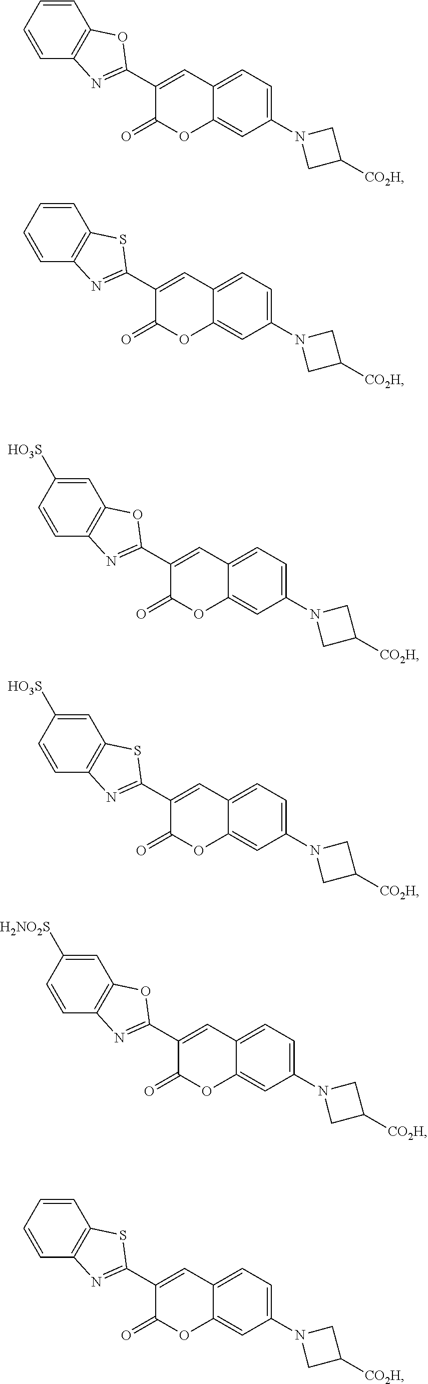 [SMILES: O=C(O)C1CN(c2ccc3cc(-c4nc5ccccc5o4)c(=O)oc3c2)C1 ; O=C(O)C1CN(c2ccc3cc(-c4nc5ccccc5s4)c(=O)oc3c2)C1 ; O=C(O)C1CN(c2ccc3cc(-c4nc5ccc(S(=O)(=O)O)cc5o4)c(=O)oc3c2)C1 ; O=C(O)C1CN(c2ccc3cc(-c4nc5ccc(S(=O)(=O)O)cc5s4)c(=O)oc3c2)C1 ; NS(=O)(=O)c1ccc2nc(-c3cc4ccc(N5CC(C(=O)O)C5)cc4oc3=O)oc2c1 ; O=C(O)C1CN(c2ccc3cc(-c4nc5ccccc5s4)c(=O)oc3c2)C1]

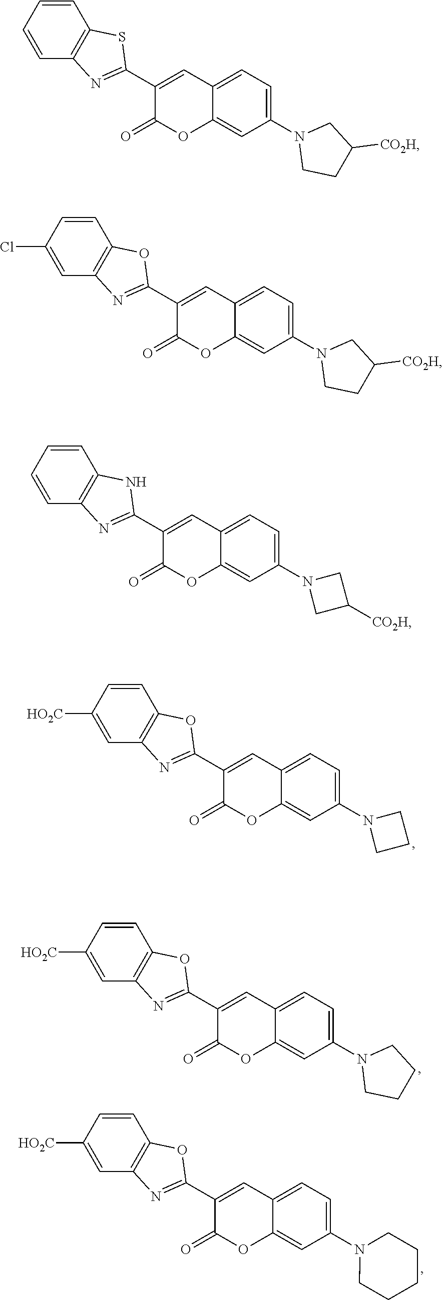 [SMILES: O=C(O)C1CCN(c2ccc3cc(-c4nc5ccccc5s4)c(=O)oc3c2)C1 ; O=C(O)C1CCN(c2ccc3cc(-c4nc5cc(Cl)ccc5o4)c(=O)oc3c2)C1 ; O=C(O)C1CN(c2ccc3cc(-c4nc5ccccc5[nH]4)c(=O)oc3c2)C1 ; O=C(O)c1ccc2oc(-c3cc4ccc(N5CCC5)cc4oc3=O)nc2c1 ; O=C(O)c1ccc2oc(-c3cc4ccc(N5CCCC5)cc4oc3=O)nc2c1 ; O=C(O)c1ccc2oc(-c3cc4ccc(N5CCCCC5)cc4oc3=O)nc2c1]

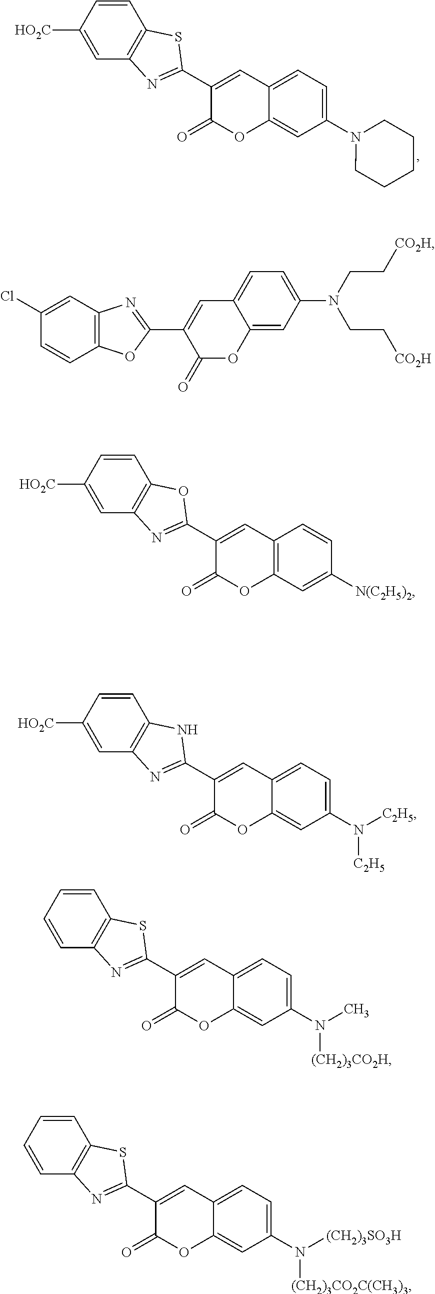

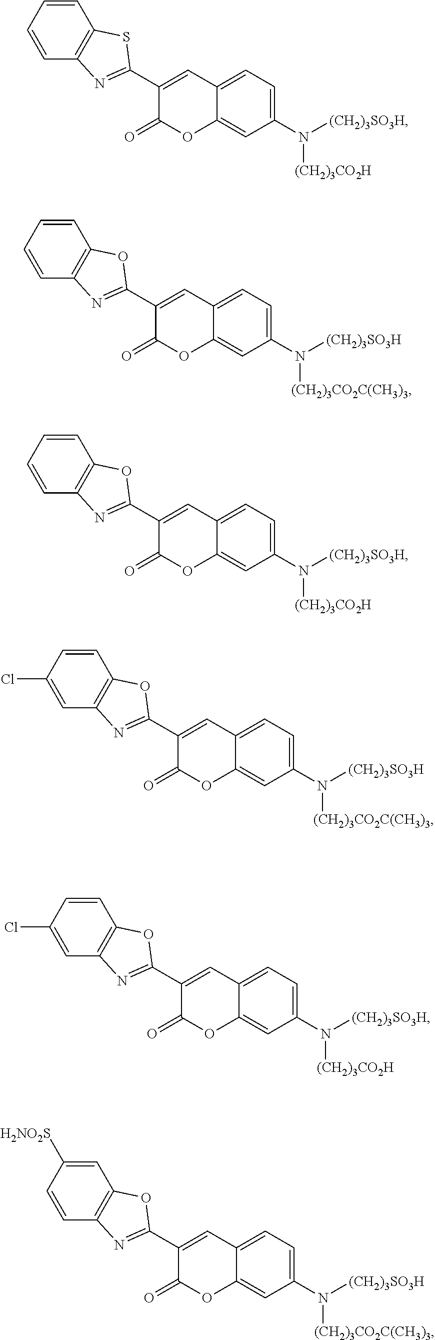

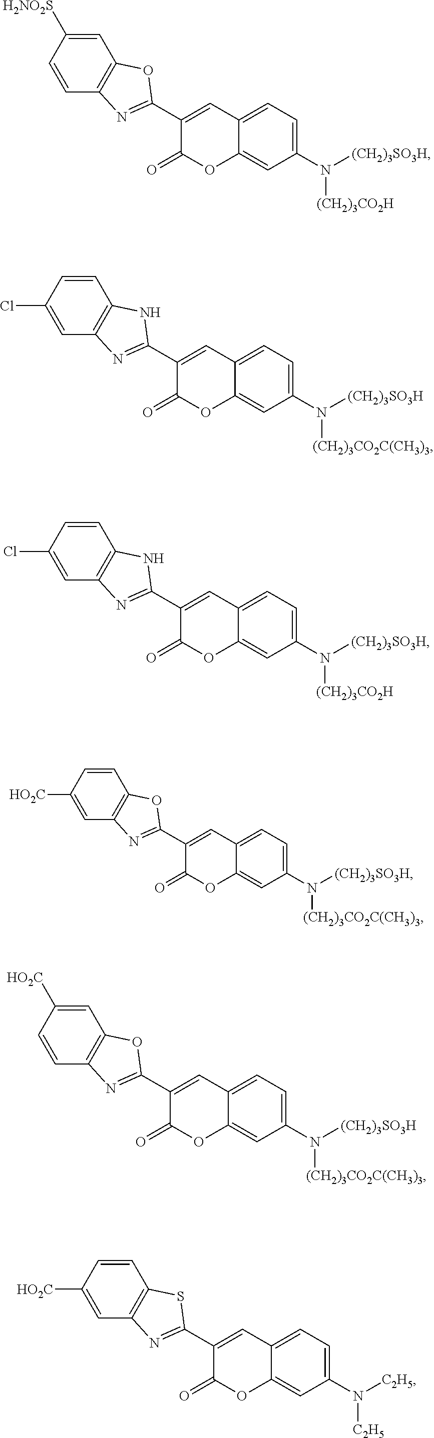

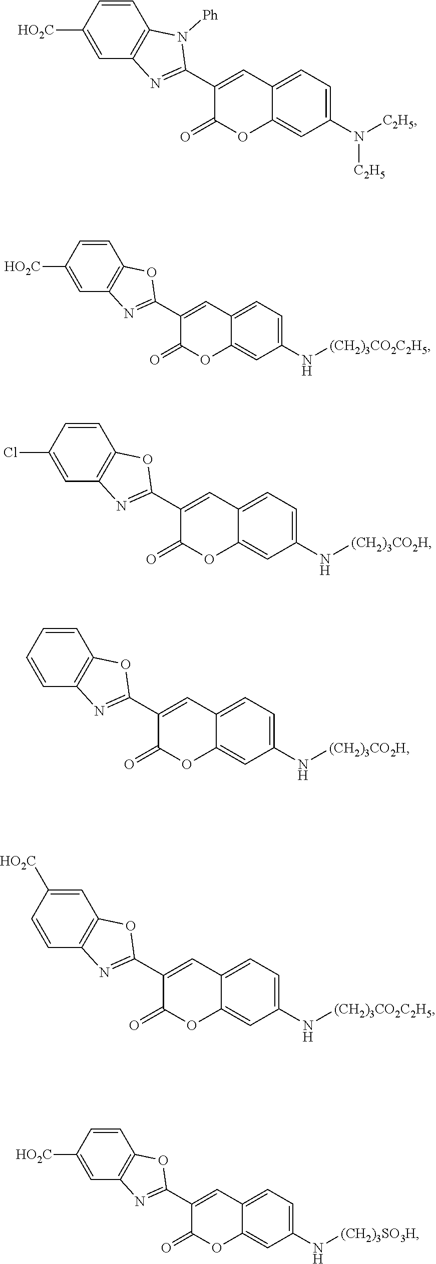

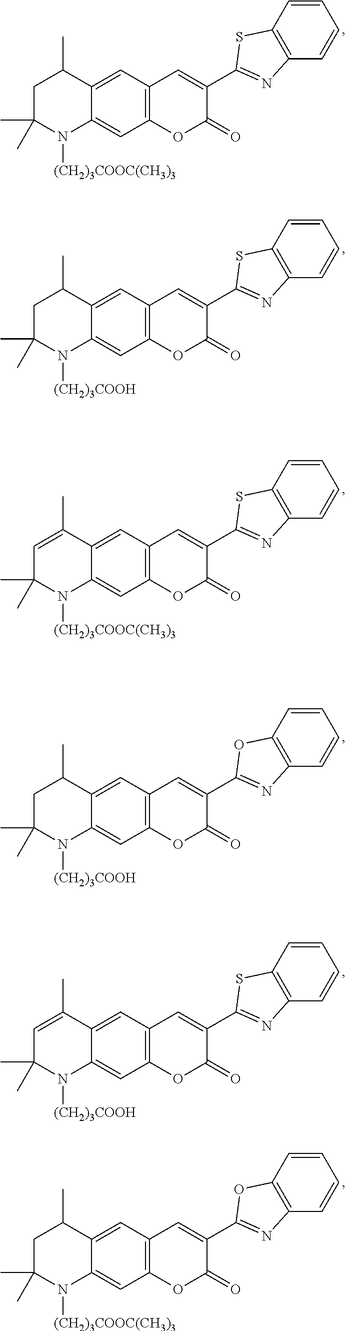

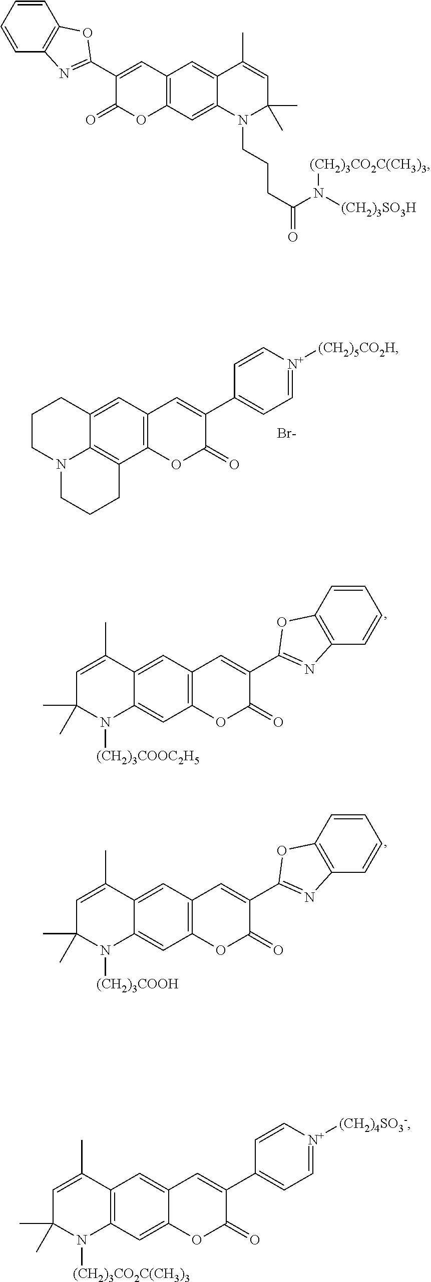

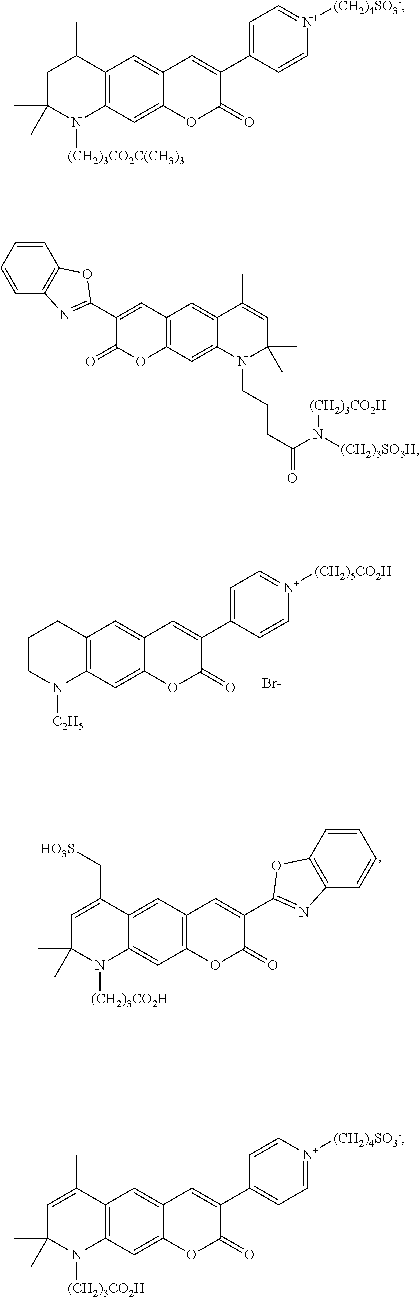

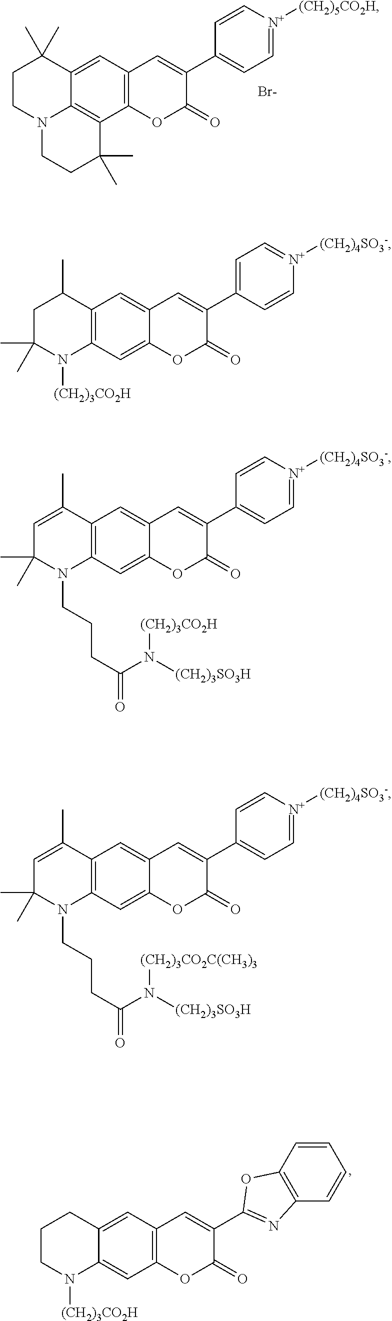

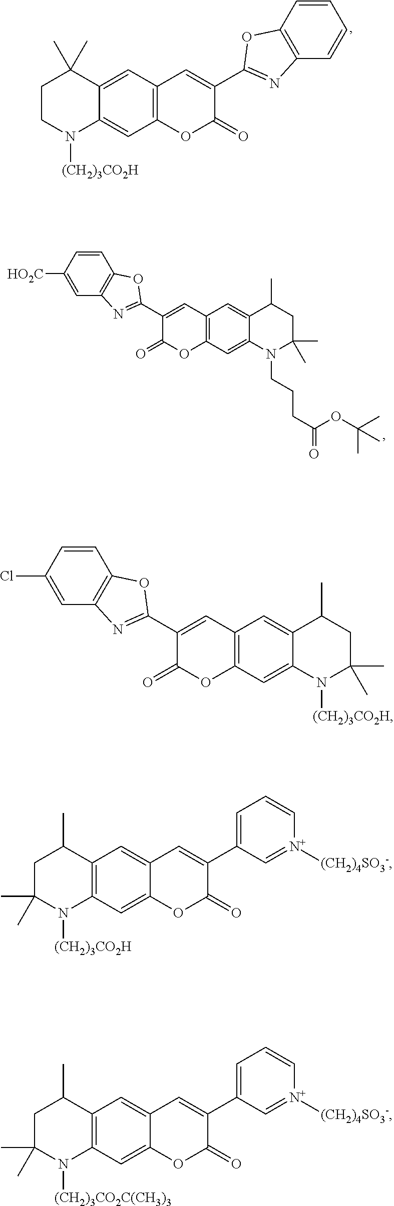

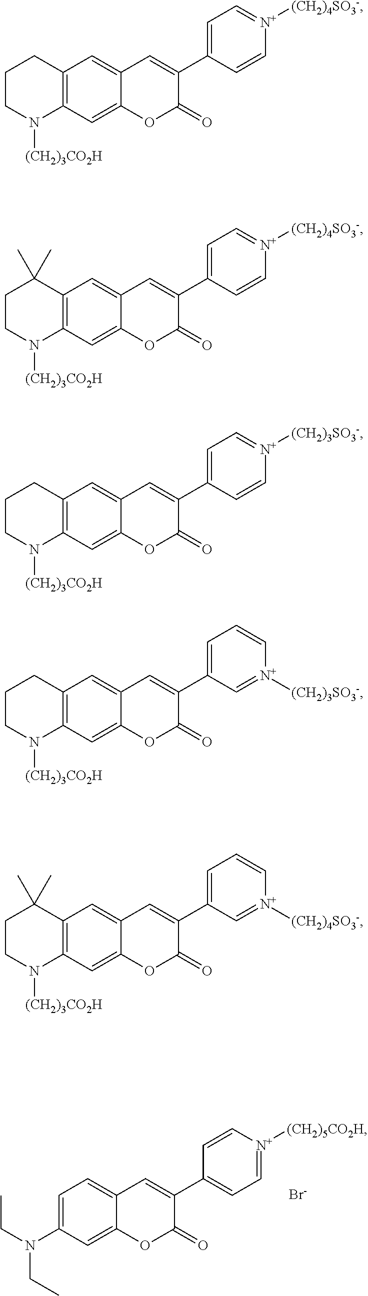

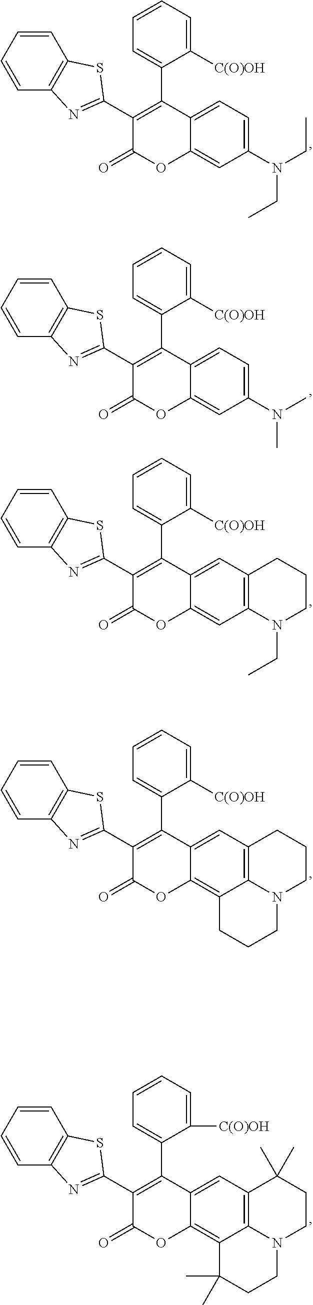

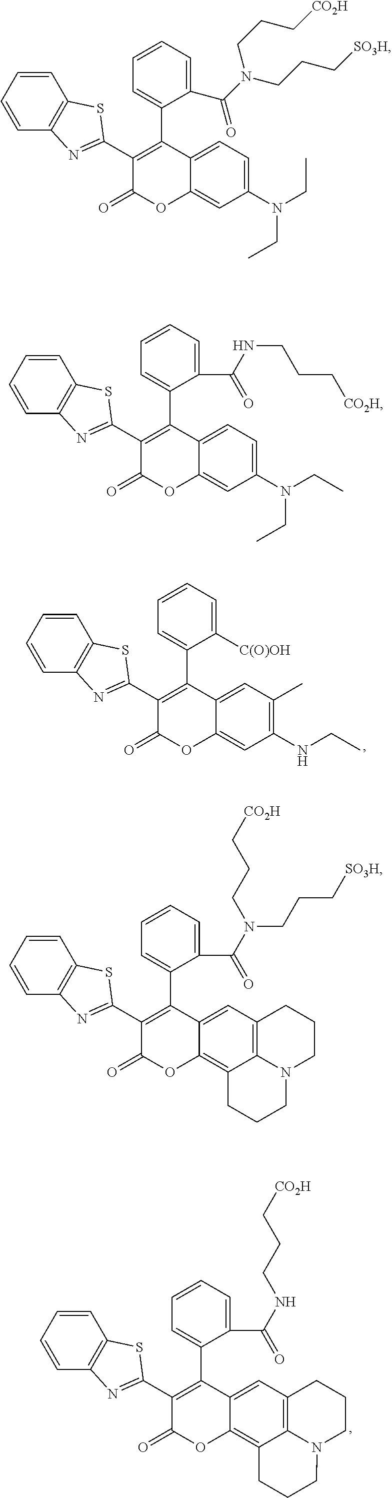

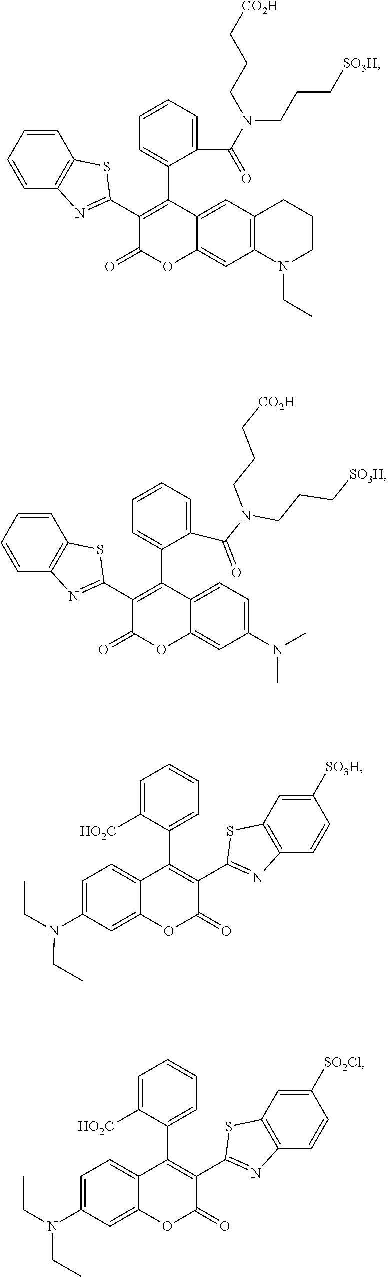 [SMILES: CCN1CCCc2cc3c(-c4ccccc4C(=O)N(CCCC(=O)O)CCCS(=O)(=O)O)c(-c4nc5ccccc5s4)c(=O)oc3cc21 ; CN(C)c1ccc2c(-c3ccccc3C(=O)N(CCCC(=O)O)CCCS(=O)(=O)O)c(-c3nc4ccccc4s3)c(=O)oc2c1 ; CCN(CC)c1ccc2c(-c3ccccc3C(=O)O)c(-c3nc4ccc(S(=O)(=O)O)cc4s3)c(=O)oc2c1 ; CCN(CC)c1ccc2c(-c3ccccc3C(=O)O)c(-c3nc4ccc(S(=O)(=O)Cl)cc4s3)c(=O)oc2c1]

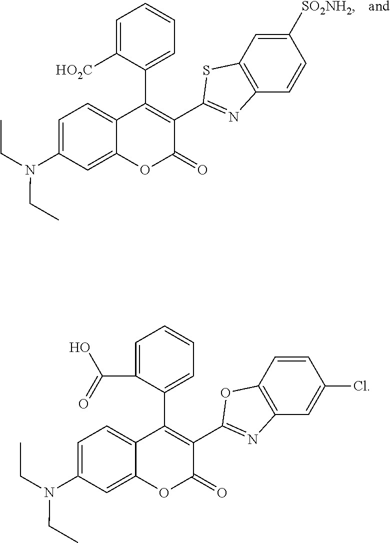 [SMILES: CCN(CC)c1ccc2c(-c3ccccc3C(=O)O)c(-c3nc4ccc(S(N)(=O)=O)cc4s3)c(=O)oc2c1 ; "and" ; CCN(CC)c1ccc2c(-c3ccccc3C(=O)O)c(-c3nc4cc(Cl)ccc4o3)c(=O)oc2c1]

In one example, the blue dye used in the sequencing method is

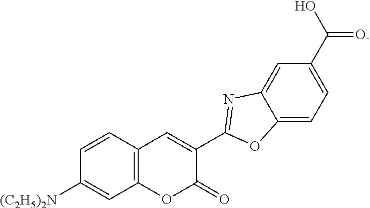

Additional exemplary dye compounds are disclosed in U.S. application Ser. No. 17/385,232, which is incorporated herein by reference.

Antioxidants/Radical Scavengers

In some embodiments, the method described herein utilizes a scan mix comprising one or more antioxidants/radical scavengers to reduce the photo damaged caused by the blue and violet excitation. In particular, the extended primer polynucleotide/target polynucleotide complex is in a buffer solution comprising one or more antioxidants during the first imaging event and the second imaging event. Useful antioxidants include but not limited to cyclooctatetraene (COT), taxifolin, quercetin, allyl thiourea, dimethyl thiourea, silibinin, ascorbic acid or a salt thereof (e.g., sodium ascorbate), polyphenolic compounds (such as 6-hydroxy-2,5,7,8-tetramethylchroman-2-carboxylic acid (Trolox), gallic acid and lower alkyl esters thereof, monomethyl ethers thereof, and combinations of lower alkyl esters and monomethyl ethers thereof, pyrogallol, and hydroquinones, such as t-butyl hydroquinone (TBHQ), 2,4,5-trihydroxybutyrophenone (THBP)), and optionally substituted derivatives and combinations thereof. In some further embodiment, the composition comprises cyclooctatetraene and quercetin, and optionally substituted derivatives and combinations thereof.

Alternatively, the blue/violet dyes described herein may also be covalently bonded to a cyclooctatetraene photo-protecting moiety. In some embodiments, a COT moiety that may be covalently bonded to a blue or violet dye described herein may comprise the structure:

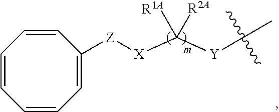

wherein each of R^{1A} and R^{2A} is independently H, hydroxyl, halogen, azido, thiol, nitro, cyano, optionally substituted amino, carboxyl, —C(O)OR^{5A}, —C(O)NR^{6A}R^{7A}, optionally substituted C₁₋₆ alkyl, optionally substituted C₁₋₆ alkoxy, optionally substituted C₁₋₆ haloalkyl, optionally substituted C₁₋₆ haloalkoxy, optionally substituted C₂₋₆ alkenyl, optionally substituted C₂₋₆ alkynyl, optionally substituted C₆₋₁₀ aryl, optionally substituted C₇₋₁₄ aralkyl, optionally substituted C₃₋₇ carbocyclyl, optionally substituted 5 to 10 membered heteroaryl, or optionally substituted 3 to 10 membered heterocyclyl;

X¹ and Y¹ are each independently a bond, —O—, —S—, —NR^{3A}—, —C(═O)—, —C(═O)—O—, —C(═O)—NR^{4A}—, —S(O)₂—, —NR^{3A}—C(═O)—NR^{4A}, —NR^{3A}—C(═S)—NR^{4A}—, optionally substituted C₁₋₆ alkylene, or optionally substituted heteroalkylene where at least one carbon atom is replaced with O, S, or N;

Z is absent, optionally substituted C₂₋₆ alkenylene, or optionally substituted C₂₋₆ alkynylene;

each of R^{3A} and R^{4A} is independently H, optionally substituted C₁₋₆ alkyl, or optionally substituted C₆₋₁₀ aryl;

R^{5A} is optionally substituted C₁₋₆ alkyl, optionally substituted C₆₋₁₀ aryl, optionally substituted C₇₋₁₄ aralkyl, optionally substituted C₃₋₇ carbocyclyl, optionally substituted 5 to 10 membered heteroaryl, or optionally substituted 3 to 10 membered heterocyclyl;

each of R^{6A} and R^{7A} is independently H, optionally substituted C₁₋₆ alkyl, optionally substituted C₆₋₁₀ aryl, optionally substituted C₇₋₁₄ aralkyl, optionally substituted C₃₋₇ carbocyclyl, optionally substituted 5 to 10 membered heteroaryl, or optionally substituted 3 to 10 membered heterocyclyl;

the carbon atom to which R^{1A} and R^{2A} are attached in

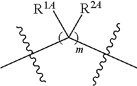

is optionally replaced with O, S, or N, provided that when said carbon atom is replaced with O or S, then R^{1A} and R^{2A} are both absent; when said carbon atom is replaced with N, then R^{2A} is absent; and m is an integral number between 0 and 10. In some embodiments, X and Y are not both a bond.

In some embodiments, the cyclooctatetraene moiety comprises the structure

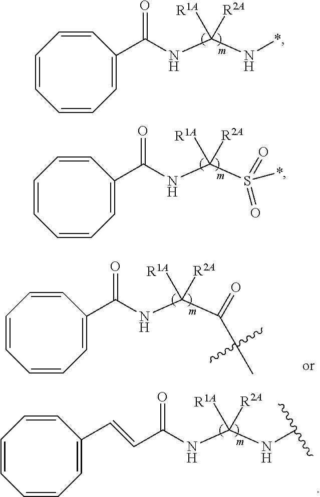

In some such embodiments, at least one of R^{1A} and R^{2A} is hydrogen. In some further embodiments, both R^{1A} and R^{2A} are hydrogen. In some other embodiments, R^{1A} is H and R^{2A} is an optionally substituted amino, carboxyl or —C(O)NR^{6A}R^{7A}. In some embodiments, m is 1, 2, 3, 4, 5, or 6, and each of R^{1A} and R^{2A} is independently hydrogen, optionally substituted amino, carboxyl, —C(O)NR^{6A}R^{7A}, or combinations thereof. In some further embodiments, when m is 2, 3, 4, 5, or 6, one R^{1A} is amino, carboxyl, or —C(O)NR^{6A}R^{7A}, and the remaining R^{1A} and R^{2A} are hydrogen. In some embodiments, at least one carbon atom to which R^{1A} and R^{2A} are attached in

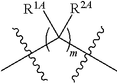

is replaced with O, S, or N. In some such embodiments, one carbon atom in

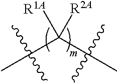

is replaced by an oxygen atom, and both R^{1A} and R^{2A} attached to said replaced carbon atom are absent. In some other embodiments, when one carbon atom in

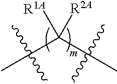

is replaced by an nitrogen atom, R^{2A} attached to said replaced carbon atom is absent, and R^{1A} attached to said replaced carbon atom is hydrogen, or C₁-6 alkyl. In any embodiments of R^{1A} and R^{2A}, when R^{1A} or R^{2A} is —C(O)NR^{6A}R^{7A}, R^{6A} and R^{7A} may be independently H, C₁₋₆ alkyl or substituted C₁₋₆ alkyl (e.g., C₁₋₆ alkyl substituted with —CO₂H, —NH₂, —SO₃H, or —SO₃⁻).

In some further embodiments, the fluorescent dyes described herein comprises a cyclooctatetraene moiety of the following structures:

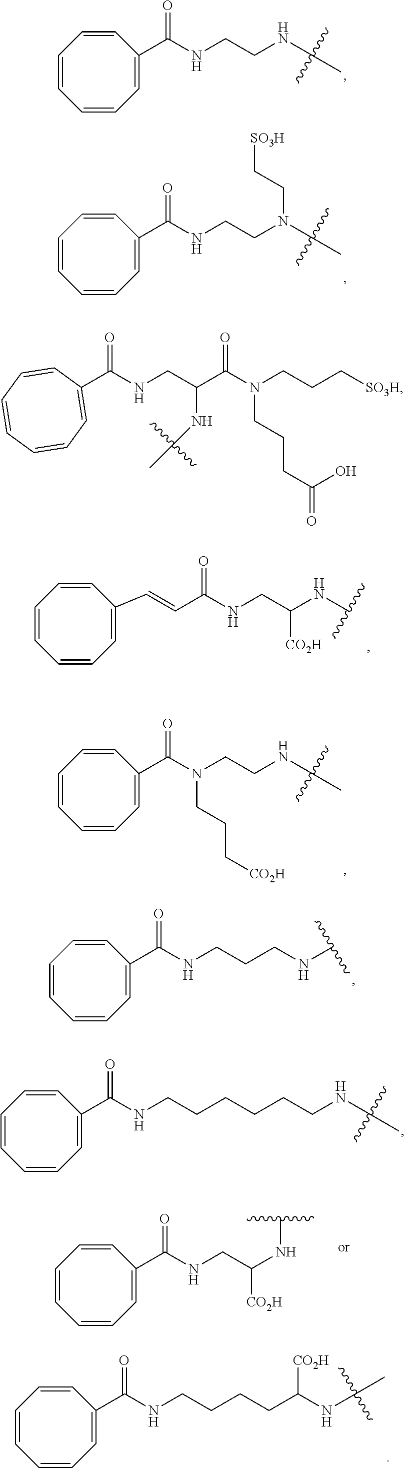

The COT moiety described herein may result from the reaction between a functional group of the fluorescent dye described herein (e.g., a carboxyl group) and an amino group of a COT derivative to form an amide bond (where the carbonyl group of the amide bond is not shown). Additional disclosure on COT related antioxidants used in SBS chemistry may be found in U.S. Publication No. 2021/0155983 A1, which is incorporated herein by reference in its entirety.
Unlabeled Nucleotides in Incorporation Mixture in Combination with Affinity Reagents

As an alternative to the embodiments described above, a second aspect of the sequence method described herein includes a secondary labeling step may be used to reduce DNA damage and photo bleaching of the dyes caused by blue/violet excitation. In some embodiments, the secondary labeling refers to a modification to the standard sequencing method where a unlabeled nucleotide in incorporated into the primer polynucleotide first, then the incorporated unlabeled nucleotide binds to an affinity reagent that is specific to the type of incorporated nucleotide, and the affinity reagents contains one or more detectable labels that can be excited by the blue or violet lights and emit signals that can be detected by the emission detection channels. Without being bound by a particular theory, the size of an affinity reagent may shield the DNA from ROS, and therefore reduce or mitigate the photodamage caused by the blue/violet lights. In some embodiments of the method described herein, one or more of the first type, the second type and the third type of nucleotide may be unlabeled. In one embodiment, each of the first type, the second type and the third type of nucleotide in the incorporation mixture is unlabeled, and the method further comprising: contacting the extended primer polynucleotide/target polynucleotide complex with a set of affinity reagents prior to the first imaging event, wherein at least one affinity reagent in the set binds specifically to the incorporated first type, second type, or third type of nucleotide. In some such embodiments, the set of affinity reagents comprises: a first affinity reagent that binds specifically to the first type of nucleotide, a second affinity reagent that binds specifically to the second type of nucleotide. In some further embodiments, the first affinity reagent comprises one or more first detectable labels that are excitable by the first excitation light source and detectable by the second emission filter, the second affinity reagent comprises one or more second detectable labels that are excitable by the second excitation light source and detectable by the second emission filter, and wherein the first detectable label is spectrally distinguishable from the second detectable label. In some such embodiments, both the first affinity reagent and the second affinity reagent bind specifically to the third type of nucleotide. In other embodiments, the set of affinity reagents further comprises a third affinity reagent that binds specifically to the third type of nucleotide, and wherein the third affinity reagent comprises one or more third detectable labels that are excitable by the first excitation light source and detectable by the first emission filter, and one or more fourth detectable labels that are excitable by the second excitation light source and detectable by the second emission filter. The third dye has similar fluorescent profile as the first dye (i.e., absorption and emission spectra) but may be different in terms of emission intensity. The fourth dye has similar fluorescent profile as the second dye (i.e., absorption and emission spectra) but may be different in terms of emission intensity.

When the affinity reagent containing detectable label(s) binds to the incorporated nucleotide, the extended primer polynucleotide/target polynucleotide complex becomes a labeled extended primer polynucleotide/target polynucleotide complex that can be detected in the first and/or second imaging event. The violet dyes and blue dyes described herein may be used in any embodiments of the modified method described herein. In addition, the labeled extended primer polynucleotide/target polynucleotide complex may be present in a scan mixture comprising one or more of the antioxidants and ROS scavengers described herein. The detectable labels in the affinity reagent(s) may also contain a covalently bonded photo-protecting moiety described herein.

Affinity Reagents

As used herein, the term “affinity reagent” refer to a macromolecule, such as a protein or an antibody, that binds specifically to an incorporated nucleotide in the extended primer polynucleotide/target polynucleotide complex. In some embodiments, the affinity reagents include antibodies (including but not limited to binding fragments of antibodies, single chain antibodies, bispecific antibodies, and the like), aptamers, knottins, affimers, or any other known agent that binds an incorporated nucleotide with a suitable specificity and affinity. Each affinity reagent described herein may have substantially higher affinity for a particular type of nucleotide than for the other types of nucleotides. Also, the affinity reagent should bind to the incorporated nucleotide at the 3′ end of a growing DNA chain (i.e., the extended primer polynucleotide), but not to a nucleotide elsewhere on the DNA chain. The affinity reagent described herein may be directly or indirectly labeled with one or more detectable labels, such as the blue dyes and violet dyes described herein. In some embodiments, the one or more detectable labels are covalently attached to the affinity reagent via a cleavable linker.

In some embodiments of the method described herein, the first type of nucleotide comprises a first hapten, and the first affinity reagent comprises a first hapten-binding partner that specifically binds to the first hapten. In some such embodiments, the second type of nucleotide comprises a second hapten, and the second affinity reagent comprises a second hapten-binding partner that specifically binds to the second hapten. Each of the first hapten and the second hapten may comprise, or be selected from the group consisting of biotin, digoxigenin, dinitrophenol or chloroalkyl group. Each of the affinity reagents comprise a specific hapten-binding partner that may be an anti-hapten antibody conjugated with one or more fluorescent moieties. In some further embodiments, the first hapten comprises a biotin moiety and the first hapten-binding partner comprises streptavidin, wherein the streptavidin comprises one or more first detectable labels. In some further embodiments, the second hapten comprises a chloroalkyl group and the second hapten-binding partner comprises HaloTag®, wherein the HaloTag® comprises one or more second detectable labels. The first or second detectable labels may be conjugated to the affinity reagent (e.g., hapten-binding partner) via one or more cleavable linker described herein. In some such embodiments, the third type of nucleotide comprises both a first hapten and a second hapten (e.g., a mixture of a third type of nucleotide comprising a first hapten and a third type of nucleotide comprising a second hapten), such that both the first affinity reagent and the second affinity reagent may bind specifically to the third type of nucleotide.

In other embodiments, when the set affinity reagent further comprises the third affinity reagent that binds specifically to the third type of nucleotide, the third type of nucleotide may comprise a third hapten and the third affinity reagent comprises a third hapten-binding partner. The third affinity reagent may be a mixture of a third affinity reagent comprising third hapten-binding partner and one or more third detectable labels, and a third affinity reagent comprising third hapten-binding partner and one or more fourth detectable labels.

For example, the first type of unlabeled nucleotide may contain a first hapten comprising a biotin moiety, whereas the first affinity reagent may comprise streptavidin conjugated with one or more first detectable labels, which is a blue dye that is excitable by a blue light source at about 450-460 nm (i.e., the first dye is a blue dye) and has an emission wavelength in the range of 480-525 nm. The second type of unlabeled nucleotide may contain a second hapten comprising a chloroalkyl group (e.g., —(CH₂)₆Cl), whereas the second affinity reagent may comprise a HaloTag® conjugated with one or more second detectable labels, which is a violet dye that is excitable by a violet light source at about 400-405 nm and has an emission wavelength in the range of 415-450 nm. The third type of unlabeled nucleotide may contain both a first hapten and a second hapten that bind to both the first affinity reagent and the second affinity reagent. In some embodiments, the fourth type of nucleotide is unlabeled and does not bind to any affinity reagent. After contacting the extended primer polynucleotide/target nucleotide complex with the set of affinity reagents, the unbounded affinity reagents are washed away. The first imaging event uses a blue light source having a wavelength of about 450-460 nm, and both the first type and the third type of nucleotides will emit a signal that can be detected or collected by an emission filter having a filter band that encompasses 480-525 nm. The second imaging event uses a violet light source having a wavelength of about 400-405 nm, and both the second type and the third type of nucleotides (through the detectable labels conjugated with the affinity reagents) will emit a signal that can be detected or collected by an emission filter having a filter band that encompasses 415-450 nm. No signal will be detected under either the first or the second imaging event for the fourth type of nucleotide. Based on the signal detection pattern described herein, the identity of the incorporated nucleotide in the extended primer polynucleotide may be determined. In a further embodiment, the incorporation mixture comprises the following: a dATP comprising a chloroalkyl group, a dTTP comprising a biotin moiety, a dCTP comprising a biotin moiety, a dCTP comprising a chloroalkyl group, and an unlabeled dGTP (dark G). Exemplary ffC comprising a biotin moiety and ffA comprising a chloroalkyl group include:

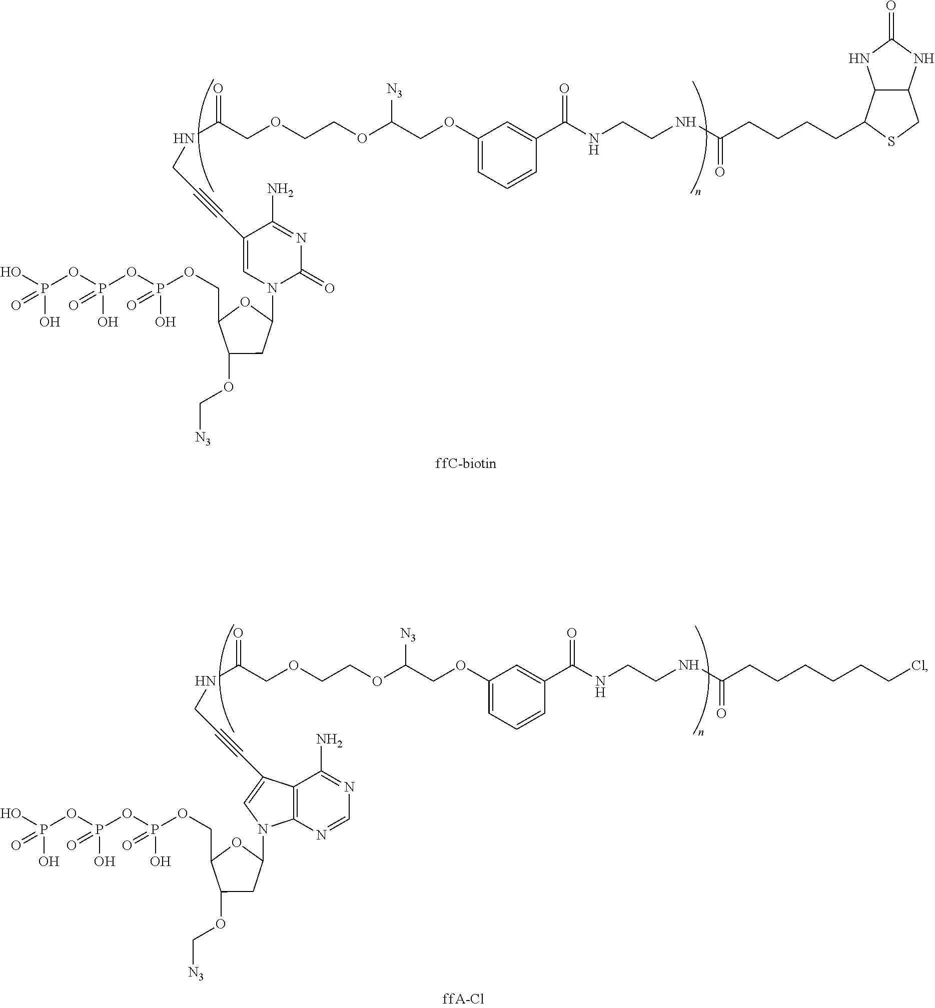

where n is 1, 2, or 3.

A third aspect of the sequencing method described herein also involves the use of secondary labeling with affinity reagent(s), but only one of the first type, the second type, or the third type of nucleotide is unlabeled. In some embodiments, the first type of nucleotide is labeled with a first detectable label, the second type of nucleotide is unlabeled, the third type of nucleotide is both unlabeled and labeled with the first detectable label, and the first detectable label is excitable by the first excitation light source and detectable by the first emission filter. The method further comprises contacting the extended primer polynucleotide with an affinity reagent prior to the first imaging event, wherein the affinity reagent binds specifically to the second type of unlabeled nucleotide and/or the third type of unlabeled nucleotide. In some such embodiments, the affinity reagent comprises one or more second detectable labels that are excitable by the second excitation light source and detectable by the second emission filter. In some further embodiments, the affinity reagent comprises streptavidin conjugated to one or more second detectable labels, and both the second type of nucleotide and the third type unlabeled nucleotide comprise a biotin moiety. In some other embodiments, the first type of nucleotide is unlabeled, the second type of nucleotide is labeled with a second detectable label, the third type of nucleotide is both unlabeled and labeled with the second detectable label, and the second detectable label is excitable by the second excitation light source and detectable by the second emission filter. The method further comprises contacting the extended primer polynucleotide with an affinity reagent prior to the first imaging event, wherein the affinity reagent binds specifically to the first type of unlabeled nucleotide and/or the third type of unlabeled nucleotide. In some such embodiments, the affinity reagent comprises one or more first detectable labels that are excitable by the first excitation light source and detectable by the first emission filter. In some further embodiments, the affinity reagent comprises streptavidin conjugated to one or more first detectable labels, and both the first type of nucleotide and the third type unlabeled nucleotide comprise a biotin moiety. In any such embodiments, the fourth type of nucleotide is unlabeled and does not bind with the affinity reagent or emit any signals during the first imaging event and the second imaging event.

As another example, the first type of nucleotide is labeled with a blue dye that is excitable by a blue light source at about 450-460 nm and has an emission wavelength in the range of 480-525 nm. The second type of nucleotide is unlabeled and comprises a biotin moiety. The affinity reagent comprises streptavidin conjugated with one or more violet dyes is excitable by a violet light source at about 400-405 nm and has an emission wavelength in the range of 415-450 nm. The third type of nucleotide is a mixture of both an unlabeled third type of nucleotide comprising a biotin moiety, and a third type of nucleotide labeled with the same blue dye as the first type of nucleotide. The fourth type of nucleotide is unlabeled and does not bind with any affinity reagent or emit any signal under the first/second imaging events. After contacting the extended primer polynucleotide/target nucleotide complex with the affinity reagent, the unbounded affinity reagent is washed away. The first imaging event uses a blue light source having a wavelength of about 450-460 nm, and both the first type and the third type of nucleotides will emit a signal that can be detected or collected by an emission filter having a filter band that encompasses 480-525 nm. The second imaging event uses a violet light source having a wavelength of about 400-405 nm, and both the second type and the third type of nucleotides (through the violet dyes attached to the streptavidin) will emit a signal that can be detected or collected by an emission filter having a filter band that encompasses 415-450 nm. No signal will be detected under either the first or the second imaging event for the fourth type of nucleotide. Based on the signal detection pattern described herein, the identity of the incorporated nucleotide in the extended primer polynucleotide may be determined. In a further embodiment, the incorporation mixture comprises the following: a dATP labeled with a blue dye, a dTTP comprising a biotin moiety, a dCTP comprising a biotin moiety, a dCTP labeled with a blue dye, and an unlabeled dGTP (dark G).

In an alternative embodiment of the third aspect of the sequencing method described herein, the first type of nucleotide is labeled with a first detectable label, the second type of nucleotide is unlabeled, the third type of nucleotide is both unlabeled and labeled with a third detectable label, and the both the first and the third detectable label are excitable by the first excitation light source and detectable by the first emission filter (e.g., the third label has similar fluorescent profile as the first label but may be different in emission intensity). The method further comprises contacting the extended primer polynucleotide with a set of affinity reagents prior to the first imaging event, wherein at least one affinity reagent binds specifically to the second type of unlabeled nucleotide, and at least one affinity reagent binds specifically to the third type of unlabeled nucleotide. In some such embodiments, the affinity reagent that specifically binds to the second type of nucleotide comprises one or more second detectable labels that are excitable by the second excitation light source and detectable by the second emission filter. The affinity reagent that specifically binds to the third type of nucleotide comprises one or more fourth detectable labels that are excitable by the second excitation light source and detectable by the second emission filter (e.g., the fourth label has similar fluorescent profile as the second label but may be different in emission intensity). In some further embodiments, the set affinity reagent may comprise streptavidin conjugated to one or more second detectable labels, and a second antibody/protein conjugated to one or more fourth detectable labels. The second type of unlabeled nucleotide comprises a biotin moiety and the third type of unlabeled nucleotide comprises a hapten that is specific to the second antibody/protein conjugated to the fourth labels.

Another alternative embodiment to the third aspect of the sequencing method described herein involves the use an incorporation mixture: the first type of nucleotide is unlabeled, the second type of nucleotide is labeled with a second detectable label, the third type of nucleotide is both unlabeled and labeled with a fourth detectable label, and both the second and the fourth detectable label are excitable by the second excitation light source and detectable by the second emission filter (e.g., the third label has similar fluorescent profile as the first label but may be different in emission intensity). The method further comprises contacting the extended primer polynucleotide with a set of affinity reagents prior to the first imaging event, wherein at least one affinity reagent binds specifically to the first type of unlabeled nucleotide, and at least one affinity reagent binds specifically to the third type of unlabeled nucleotide. In some such embodiments, the affinity reagent that specifically binds to the first type of nucleotide comprises one or more first detectable labels. The affinity reagent that specifically binds to the third type of nucleotide comprises one or more third detectable labels. Both the first and the third detectable labels are excitable by the first excitation light source and detectable by the first emission filter (e.g., the third label has similar fluorescent profile as the first label but may be different in emission intensity).

In any embodiments of the method described herein, the nucleotides in the mixture in step (a) comprise four different types of nucleotide (A, C, G, and T or U), or non-natural nucleotide analogs thereof. In further embodiments, the four different types of nucleotides are dATP, dCTP, dGTP and dTTP or dUTP, or non-natural nucleotide analogs thereof. In some further embodiments, three of the four types of nucleotide are each labeled with a detectable label, and one of the nucleotide is not labeled with a fluorophore, or is labeled with a fluorophore but cannot be exited and emits a signal in either the first imaging or the second imaging event. In other embodiments, the detectable label in the one, two or three of the four types of nucleotide are added using a secondary labeling step described herein, in which an unlabeled nucleotide is first incorporated into the primer polynucleotide, then an affinity reagent specific to the type of nucleotide incorporate is introduced to the primer polynucleotide, wherein the affinity reagent contains one or more detectable labels that can emit signal(s) during the first and/or the second imaging event. In further embodiments, each of the four types of nucleotide in the incorporation mixture contains a 3′ hydroxyl blocking group. Such 3′ hydroxyl blocking group ensures that only a single base can be added by a polymerase to the 3′ end of the primer polynucleotide. After incorporation of a nucleotide in step (b), the remaining unincorporated nucleotides are washed away.

In some embodiments of the method described herein, the method further includes step (e): removing the 3′ hydroxyl blocking group from the incorporated nucleotide after the second imaging event, and prior to the next sequencing cycle. In further embodiments, any detectable label attached to the incorporated nucleotide (either directly to incorporated nucleotide via a cleavable linker; or indirectly via an affinity reagent) is also removed prior to the next sequencing cycle. In some such embodiments, the detectable label and the 3′ hydroxy blocking group are removed in a single step (e.g., under the same chemical reaction condition). In other embodiments, the label and the 3′ hydroxy blocking group are removed in two separate steps (e.g., the label and the 3′ blocking group are removed under two separate chemical reaction conditions). In some further embodiments, a post cleavage washing step is used after the label and the 3′ blocking group are removed. In further embodiments, steps (a) through (e) are performed in repeated cycles (e.g., at least 30, 50, 100, 150, 200, 250, 300, 400, or 500 times) and the method further comprises sequentially determining the sequence of at least a portion of the single-stranded target polynucleotide based on the identity of each sequentially incorporated nucleotides. In some such embodiments, steps (a) through (e) are repeated at least 50 cycles. In some further embodiments, the incorporation of the nucleotide from the incorporation mixture is performed by a polymerase (e.g., a DNA polymerase). Exemplary polymerases include but not limited to Pol 812, Pol 1901, Pol 1558 or Pol 963. The amino acid sequences of Pol 812, Pol 1901, Pol 1558 or Pol 963 DNA polymerases are described, for example, in U.S. Patent Publication Nos. 2020/0131484 A1 and 2020/0181587 A1, both of which are incorporated by reference herein.

In some embodiments of the method described herein, each of the first excitation light source used in the first imaging event and the second excitation light source used in the second imaging event comprises a laser, a light-emitting diode (LED), or a combination thereof.

In some embodiments, the combination of emission detection from the first imaging event and the second imaging event are processed by image analysis software to determine the identity of the bases are incorporated at each immobilized primer polynucleotide/target polynucleotide complex position. In some such embodiments, the image analysis is processed after repeated cycles of incorporation (after at least 50, 100, 150, 200, 250 or 300 runs).

In any embodiments of the method described herein, the single-stranded target polynucleotide may be immobilized to a solid support. In some such embodiment, the solid support comprises a plurality of immobilized single-stranded target polynucleotides. The primer polynucleotide is complementary to at least a portion of a target polynucleotide. In some such embodiments, the primer polynucleotide is hybridized to at least a portion of the target polynucleotide to form a primer polynucleotide/target polynucleotide complex. The solid support may comprise clustered primer polynucleotide/target polynucleotide complexes. In some embodiments, the solid support comprises a flowcell, for example, a patterned flowcell comprising a plurality of nanowells, each is separate from another. In some further embodiment, each nanowell comprises one immobilized cluster therein. In some embodiments, the density of the nanowells on the patterned flow cell is from about 100K/mm² to about 500K/mm², about 200K/mm² to about 400K/mm², or about 250K/mm² to about 350K/mm². In some embodiments, the density of the immobilized single stranded target polynucleotides (or the clusters formed from hybridization with the primer polynucleotides) on the solid support is from about 80K/mm² to about 400K/mm², about 100K/mm² to about 300K/mm², or about 150K/mm² to about 250K/mm². In some embodiments, the sequencing method described herein allows for up to 10%, 15%, 20%, 25%, 30%, 35%, 40%, 45% or 50% increase in cluster density compared to the two-channel sequencing methods using red/green or green/blue excitation with similar or comparable optical resolution.

Additional illustrative embodiments of the methods are described below.

In a specific embodiment, a synthetic step is carried out and may optionally comprise incubating a template or target polynucleotide strand with a reaction mixture comprising fluorescently labeled nucleotides of the disclosure. A polymerase can also be provided under conditions which permit formation of a phosphodiester linkage between a free 3′ OH group on a polynucleotide strand annealed to the template or target polynucleotide strand and a 5′ phosphate group on the labeled nucleotide. Thus, a synthetic step can include formation of a polynucleotide strand as directed by complementary base-pairing of nucleotides to a template/target strand.

In all embodiments of the methods, the detection step may be carried out while the polynucleotide strand into which the labeled nucleotides are incorporated is annealed to a target strand, or after a denaturation step in which the two strands are separated. Further steps, for example chemical or enzymatic reaction steps or purification steps, may be included between the synthetic step and the detection step. In particular, the polynucleotide strand incorporating the labeled nucleotide(s) may be isolated or purified and then processed further or used in a subsequent analysis. By way of example, polynucleotide strand incorporating the labeled nucleotide(s) as described herein in a synthetic step may be subsequently used as labeled probes or primers. In other embodiments, the product of the synthetic step set forth herein may be subject to further reaction steps and, if desired, the product of these subsequent steps purified or isolated.

Suitable conditions for the synthetic step will be well known to those familiar with standard molecular biology techniques. In one embodiment, a synthetic step may be analogous to a standard primer extension reaction using nucleotide precursors, including the labeled nucleotides as described herein, to form an extended polynucleotide strand (primer polynucleotide strand) complementary to the target strand in the presence of a suitable polymerase enzyme. In other embodiments, the synthetic step may itself form part of an amplification reaction producing a labeled double stranded amplification product comprised of annealed complementary strands derived from copying of the primer target polynucleotide strands. Other exemplary synthetic steps include nick translation, strand displacement polymerization, random primed DNA labeling, etc. A particularly useful polymerase enzyme for a synthetic step is one that is capable of catalyzing the incorporation of the labeled nucleotides as set forth herein. A variety of naturally occurring or mutant/modified polymerases can be used. By way of example, a thermostable polymerase can be used for a synthetic reaction that is carried out using thermocycling conditions, whereas a thermostable polymerase may not be desired for isothermal primer extension reactions. Suitable thermostable polymerases which are capable of incorporating the labeled nucleotides according to the disclosure include those described in WO 2005/024010 or WO 06/120433, each of which is incorporated herein by reference. In synthetic reactions which are carried out at lower temperatures such as 37° C., polymerase enzymes need not necessarily be thermostable polymerases, therefore the choice of polymerase will depend on a number of factors such as reaction temperature, pH, strand-displacing activity and the like.

In specific non-limiting embodiments, the disclosure encompasses methods of nucleic acid sequencing, re-sequencing, whole genome sequencing, single nucleotide polymorphism scoring, any other application involving the detection of the modified nucleotide or nucleoside labeled with dyes set forth herein when incorporated into a polynucleotide.

In a particular embodiment the disclosure provides use of labeled nucleotides comprising dye moiety according to the disclosure in a polynucleotide sequencing-by-synthesis reaction. Sequencing-by-synthesis generally involves sequential addition of one or more nucleotides or oligonucleotides to a growing polynucleotide chain in the 5′ to 3′ direction using a polymerase or ligase in order to form an extended polynucleotide chain complementary to the template/target nucleic acid to be sequenced. The identity of the base present in one or more of the added nucleotides can be determined in a detection or “imaging” step. The identity of the added base may be determined after each nucleotide incorporation step. The sequence of the template may then be inferred using conventional Watson-Crick base-pairing rules. The use of the nucleotides labeled with dyes set forth herein for determination of the identity of a single base may be useful, for example, in the scoring of single nucleotide polymorphisms, and such single base extension reactions are within the scope of this disclosure.

In an embodiment of the present disclosure, the sequence of a target polynucleotide is determined by detecting the incorporation of one or more nucleotides into a nascent strand complementary to the target polynucleotide to be sequenced through the detection of fluorescent label(s) attached to the incorporated nucleotide(s). Sequencing of the target polynucleotide can be primed with a suitable primer (or prepared as a hairpin construct which will contain the primer as part of the hairpin), and the nascent chain is extended in a stepwise manner by addition of nucleotides to the 3′ end of the primer in a polymerase-catalyzed reaction.

In particular embodiments, each of the different nucleotide triphosphates (A, T, G and C) may be labeled with a unique fluorophore and also comprises a blocking group at the 3′ position to prevent uncontrolled polymerization. Alternatively, one of the four nucleotides may be unlabeled (dark). The polymerase enzyme incorporates a nucleotide into the nascent chain complementary to the template/target polynucleotide, and the blocking group prevents further incorporation of nucleotides. Any unincorporated nucleotides can be washed away and the fluorescent signal pattern from each incorporated nucleotide can be “read” optically by suitable means, such as a charge-coupled device using light excitation and suitable emission filters. The 3′ blocking group and fluorescent dye compounds can then be removed (cleaved) (simultaneously or sequentially) to expose the nascent chain for further nucleotide incorporation. Typically, the identity of the incorporated nucleotide will be determined after each incorporation step, but this is not strictly essential. Similarly, U.S. Pat. No. 5,302,509 (which is incorporated herein by reference) discloses a method to sequence polynucleotides immobilized on a solid support.

The method, as exemplified above, utilizes the incorporation of 3′ blocked nucleotides A, G, C, and T into a growing strand complementary to the immobilized polynucleotide, in the presence of DNA polymerase. The polymerase incorporates a base complementary to the target polynucleotide but is prevented from further addition by the 3′ hydroxyl blocking group. The label of the incorporated nucleotide can then be determined, and the blocking group removed by chemical cleavage to allow further polymerization to occur. The nucleic acid template to be sequenced in a sequencing-by-synthesis reaction may be any polynucleotide that it is desired to sequence. The nucleic acid template for a sequencing reaction will typically comprise a double stranded region having a free 3′ OH group that serves as a primer or initiation point for the addition of further nucleotides in the sequencing reaction. The region of the template to be sequenced will overhang this free 3′ OH group on the complementary strand. The overhanging region of the template to be sequenced may be single stranded but can be double-stranded, provided that a “nick is present” on the strand complementary to the target strand to be sequenced to provide a free 3′ OH group for initiation of the sequencing reaction. In such embodiments, sequencing may proceed by strand displacement. In certain embodiments, a primer bearing the free 3′ OH group may be added as a separate component (e.g., a short oligonucleotide) that hybridizes to a single-stranded region of the template to be sequenced. Alternatively, the primer and the template strand to be sequenced may each form part of a partially self-complementary nucleic acid strand capable of forming an intra-molecular duplex, such as for example a hairpin loop structure. Hairpin polynucleotides and methods by which they may be attached to solid supports are disclosed in PCT Publication Nos. WO 01/57248 and WO 2005/047301, each of which is incorporated herein by reference. Nucleotides can be added successively to a growing primer, resulting in synthesis of a polynucleotide chain in the 5′ to 3′ direction. The nature of the base which has been added may be determined, particularly but not necessarily after each nucleotide addition, thus providing sequence information for the nucleic acid template. Thus, a nucleotide is incorporated into a nucleic acid strand (or polynucleotide) by joining of the nucleotide to the free 3′ OH group of the nucleic acid strand via formation of a phosphodiester linkage with the 5′ phosphate group of the nucleotide.

The nucleic acid template to be sequenced may be DNA or RNA, or even a hybrid molecule comprised of deoxynucleotides and ribonucleotides. The nucleic acid template may comprise naturally occurring and/or non-naturally occurring nucleotides and natural or non-natural backbone linkages, provided that these do not prevent copying of the template in the sequencing reaction.

In certain embodiments, the target polynucleotides to be sequenced may be attached to a solid support via any suitable linkage method known in the art, for example via covalent attachment. In certain embodiments, target polynucleotides may be attached directly to a solid support (e.g., a silica-based support). However, in other embodiments of the disclosure the surface of the solid support may be modified in some way so as to allow either direct covalent attachment of target polynucleotides, or to immobilize the target polynucleotides through a hydrogel or polyelectrolyte multilayer, which may itself be non-covalently attached to the solid support.

Arrays in which polynucleotides have been directly attached to a support (for example, silica-based supports such as those disclosed in WO 00/06770 (incorporated herein by reference), wherein polynucleotides are immobilized on a glass support by reaction between a pendant epoxide group on the glass with an internal amino group on the polynucleotide. In addition, polynucleotides can be attached to a solid support by reaction of a sulfur-based nucleophile with the solid support, for example, as described in WO 2005/047301 (incorporated herein by reference). A still further example of solid-supported target polynucleotides is where the template polynucleotides are attached to hydrogel supported upon silica-based or other solid supports, for example, as described in WO 00/31148, WO 01/01143, WO 02/12566, WO 03/014392, U.S. Pat. No. 6,465,178, and WO 00/53812, each of which is incorporated herein by reference.

A particular surface to which template polynucleotides may be immobilized is a polyacrylamide hydrogel. Polyacrylamide hydrogels are described in the references cited above and in WO 2005/065814, which is incorporated herein by reference. Specific hydrogels that may be used include those described in WO 2005/065814 and U.S. Pub. No. 2014/0079923. In one embodiment, the hydrogel is PAZAM (poly(N-(5-azidoacetamidylpentyl) acrylamide-co-acrylamide)).

DNA template molecules can be attached to beads or microparticles, for example, as described in U.S. Pat. No. 6,172,218 (which is incorporated herein by reference). Attachment to beads or microparticles can be useful for sequencing applications. Bead libraries can be prepared where each bead contains different DNA sequences. Exemplary libraries and methods for their creation are described in Nature, 437, 376-380 (2005); Science, 309, 5741, 1728-1732 (2005), each of which is incorporated herein by reference. Sequencing of arrays of such beads using nucleotides set forth herein is within the scope of the disclosure.

Template(s) that are to be sequenced may form part of an “array” on a solid support, in which case the array may take any convenient form. Thus, the method of the disclosure is applicable to all types of high-density arrays, including single-molecule arrays, clustered arrays, and bead arrays. Nucleotides labeled with dye compounds of the present disclosure may be used for sequencing templates on essentially any type of array, including but not limited to those formed by immobilization of nucleic acid molecules on a solid support.

However, nucleotides labeled with dye compounds of the disclosure are particularly advantageous in the context of sequencing of clustered arrays. In clustered arrays, distinct regions on the array (often referred to as sites, or features) comprise multiple polynucleotide template molecules. Generally, the multiple polynucleotide molecules are not individually resolvable by optical means and are instead detected as an ensemble. Depending on how the array is formed, each site on the array may comprise multiple copies of one individual polynucleotide molecule (e.g., the site is homogenous for a particular single- or double-stranded nucleic acid species) or even multiple copies of a small number of different polynucleotide molecules (e.g., multiple copies of two different nucleic acid species). Clustered arrays of nucleic acid molecules may be produced using techniques generally known in the art. By way of example, WO 98/44151 and WO 00/18957, each of which is incorporated herein by reference, describe methods of amplification of nucleic acids wherein both the template and amplification products remain immobilized on a solid support in order to form arrays comprised of clusters or “colonies” of immobilized nucleic acid molecules. The nucleic acid molecules present on the clustered arrays prepared according to these methods are suitable templates for sequencing using nucleotides labeled with dye compounds of the disclosure.

Nucleotides labeled with dye compounds of the present disclosure are also useful in sequencing of templates on single molecule arrays. The term “single molecule array” or “SMA” as used herein refers to a population of polynucleotide molecules, distributed (or arrayed) over a solid support, wherein the spacing of any individual polynucleotide from all others of the population is such that it is possible to individually resolve the individual polynucleotide molecules. The target nucleic acid molecules immobilized onto the surface of the solid support can thus be capable of being resolved by optical means in some embodiments. This means that one or more distinct signals, each representing one polynucleotide, will occur within the resolvable area of the particular imaging device used.

Single molecule detection may be achieved wherein the spacing between adjacent polynucleotide molecules on an array is at least 100 nm, more particularly at least 250 nm, still more particularly at least 300 nm, even more particularly at least 350 nm. Thus, each molecule is individually resolvable and detectable as a single molecule fluorescent point, and fluorescence from said single molecule fluorescent point also exhibits single step photobleaching.

The terms “individually resolved” and “individual resolution” are used herein to specify that, when visualized, it is possible to distinguish one molecule on the array from its neighboring molecules. Separation between individual molecules on the array will be determined, in part, by the particular technique used to resolve the individual molecules. The general features of single molecule arrays will be understood by reference to published applications WO 00/06770 and WO 01/57248, each of which is incorporated herein by reference. Although one use of the labeled nucleotides of the disclosure is in sequencing-by-synthesis reactions, the utility of the such nucleotides is not limited to such methods. In fact, the labeled nucleotides described herein may be used advantageously in any sequencing methodology which requires detection of fluorescent labels attached to nucleotides incorporated into a polynucleotide.

### Kits

Some aspects of the present disclosure relate to kits for the blue/violet two-channel sequencing methods described herein. In some embodiments, a kit for sequencing application, comprising:

a first type of nucleotide labeled with a first detectable label;

a second type of nucleotide labeled with a second detectable label; and

a third type of nucleotide labeled with the first detectable label and the second detectable label;

wherein the first detectable label and the second detectable label are spectrally distinguishable from one another, the first detectable label is excitable by a first light source and detectable by a first emission filter, and the second detectable label is excitable by a second light source and detectable by a second emission filter;

wherein one of the first excitation light source and the second excitation light source has a wavelength of about 350 nm to about 410 nm, and the other one of the first excitation light source and the second excitation light source has a wavelength of about 450 nm to about 460 nm; and

wherein one of the first emission filter and the second emission filter has a detection wavelength of about 415 nm to about 450 nm, and the other one of the first emission filter and the second emission filter has a detection wavelength of about 480 nm to about 525 nm. In some embodiments, the third type of nucleotide is a mixture of a third type of nucleotide labeled with the first detectable label and a third type of nucleotide labeled with the second detectable label. As a specific example, the kit may comprise a dATP labeled with a blue dye, a dTTP comprising a biotin moiety, a dCTP comprising a biotin moiety, a dCTP labeled with a blue dye, and an unlabeled dGTP (dark G).

In a second aspect of the kits for sequencing application, the kit comprising:
a first type of nucleotide labeled with a first detectable label;
a second type of nucleotide labeled with a second detectable label; and
a third type of nucleotide labeled with a third detectable label and a fourth detectable label;
wherein the first detectable label and the second detectable label are spectrally distinguishable from one another, the first detectable label is excitable by a first light source and detectable by a first emission filter, and the second detectable label is excitable by a second light source and detectable by a second emission filter;
wherein the third detectable label and the fourth detectable label are spectrally distinguishable from one another, the third detectable label is excitable by the first light source and detectable by the first emission filter, and the fourth detectable label is excitable by the second light source and detectable by the second emission filter;
wherein one of the first excitation light source and the second excitation light source has a wavelength of about 350 nm to about 410 nm, and the other one of the first excitation light source and the second excitation light source has a wavelength of about 450 nm to about 460 nm; and

wherein one of the first emission filter and the second emission filter has a detection wavelength of about 415 nm to about 450 nm, and the other one of the first emission filter and the second emission filter has a detection wavelength of about 480 nm to about 525 nm. In some embodiments, the third type of nucleotide is a mixture of a third type of nucleotide labeled with the third detectable label and a third type of nucleotide labeled with the fourth detectable label.

In a third aspect of the kits described herein, one or more types of nucleotide may be unlabeled. In some instances, a kit for sequencing application, comprising:

a first type of unlabeled nucleotide;

a second type of unlabeled nucleotide;

a third type of unlabeled nucleotide; and

a set of affinity reagents comprising: a first affinity reagent that binds specifically to the first type of unlabeled nucleotide; and a second affinity reagent that binds specifically to the second type of unlabeled nucleotide; wherein the first affinity reagent comprises one or more first detectable labels that are excitable by a first excitation light source and detectable by a first emission filter, the second affinity reagent comprises one or more second detectable labels that are excitable by a second excitation light source and detectable by a second emission filter, and wherein the first detectable label is spectrally distinguishable from the second detectable label. In some such embodiments, the first type of nucleotide comprises a first hapten, and the first affinity reagent comprises a first hapten-binding partner that specifically binds to the first hapten. In further embodiments, the first hapten comprises or consists of a biotin moiety and the first hapten-binding partner comprises or consists of streptavidin, wherein the streptavidin is conjugated with one or more first detectable labels, optionally through a cleavable linker. In some further embodiments, the second type of nucleotide comprises a second hapten, and the second affinity reagent comprises a second hapten-binding partner that specifically binds to the second hapten. In further embodiments, the second hapten comprises a chloroalkyl group (e.g., —(CH₂)₆Cl) and the second hapten-binding partner comprises or consists of HaloTag®, wherein the HaloTag® is conjugated with one or more second detectable labels, optionally through a cleavable linker. In some further embodiments, the third type of nucleotide comprises a mixture of the first hapten and the second hapten. Additional haptens may also be used in the unlabeled nucleotide described herein, including but not limited to digoxigenin and dinitrophenol. The corresponding affinity reagents will contain an anti-digoxigenin binding partner or anti-dinitrophenol binding partner. In some embodiments, both the first affinity reagent and the second affinity reagent bind specifically to the third type of unlabeled nucleotide. In other embodiments, the set of affinity reagents further comprises a third affinity reagent that binds specifically to the third type of nucleotide, and wherein the third affinity reagent comprises one or more third detectable labels that are excitable by the first excitation light source and detectable by the first emission filter, and one or more fourth detectable labels that are excitable by the second excitation light source and detectable by the second emission filter. In some such embodiments, the third type of nucleotide comprises a first hapten and the third affinity reagent comprises a third hapten-binding partner. In further embodiments, the third affinity reagent comprises a mixture of a third affinity reagent comprising a third hapten-binding partner and one or more third detectable labels, and a third affinity reagent comprising a third hapten-binding partner and one or more fourth detectable labels.

In a fourth aspect of the kit for sequencing application, the kit comprising:

a first type of nucleotide either unlabeled or labeled with a first detectable label;

a second type of nucleotide either unlabeled or labeled with a second detectable label, wherein one of the first type of nucleotide and the second type of nucleotide is unlabeled;

a third type of unlabeled nucleotide, and a third type of nucleotide labeled with the same detectable label as either the first or the second type of nucleotide, wherein the first detectable label and the second detectable label are spectrally distinguishable from one another, the first detectable label is excitable by a first light source and detectable by a first emission filter, and the second detectable label is excitable by a second light source and detectable by a second emission filter; and

an affinity reagent comprising either a first affinity reagent that binds specifically to the third type of unlabeled nucleotide and the first type of nucleotide if the first type of nucleotide is unlabeled, or a second affinity reagent that binds specifically to the third type of unlabeled nucleotide and the second type of nucleotide if the second type of nucleotide is unlabeled, wherein the first affinity reagent comprises one or more first detectable labels and the second affinity reagent comprises one or more second detectable labels. In some embodiments, the first type of nucleotide is unlabeled, the second type of nucleotide is labeled with a second detectable label, the third type of nucleotide is both unlabeled and labeled with a second detectable label, and the affinity reagent is the first affinity reagent that binds specifically to the first type of nucleotide and the third type of unlabeled nucleotide, and wherein the first affinity reagent comprises one or more first detectable labels. In other embodiments, the first type of nucleotide is labeled with a first detectable label, the second type of nucleotide is unlabeled, the third type of nucleotide is both unlabeled and labeled with a second detectable label, and the affinity reagent is the second affinity reagent that binds specifically to the second type of nucleotide and the third type of unlabeled nucleotide, and wherein the second affinity reagent comprises one or more second detectable labels. In further embodiments, when either the first type or the second type of nucleotide is unlabeled, such unlabeled nucleotide independently comprises a hapten. In further embodiments, the affinity reagent comprises a hapten-binding partner that specifically binds to the hapten in the unlabeled nucleotide. In one example, the hapten comprises a biotin moiety and the hapten binding partner comprises streptavidin. Such streptavidin is conjugated with one or more first detectable labels, optionally through a cleavable linker. In another example, the hapten comprises a chloroalkyl group and the hapten binding partner comprises HaloTag®. The HaloTag® is conjugated with one or more second detectable labels, optionally through a cleavable linker.

As an alternative to the fourth aspect of the kit described herein, the kit may comprise:

a first type of nucleotide either unlabeled or labeled with a first detectable label;

a second type of nucleotide either unlabeled or labeled with a second detectable label, wherein one of the first type of nucleotide and the second type of nucleotide is unlabeled;

a third type of nucleotide, which may comprise: (i) a mixture of unlabeled third type of nucleotide and third type of nucleotide labeled with a third detectable label when the second type nucleotide is unlabeled; or (ii) a mixture of unlabeled third type of nucleotide and third type of nucleotide labeled with a fourth detectable label when the first type nucleotide is unlabeled;

wherein the first detectable label and the second detectable label are spectrally distinguishable from one another, both the first and the third detectable labels are excitable by a first light source and detectable by a first emission filter, and both the second and the fourth detectable labels are excitable by a second light source and detectable by a second emission filter; and

a set of affinity reagent, which may comprise: (iii) a mixture of a first affinity reagent that binds specifically to the first type of nucleotide when the first type of nucleotide is unlabeled, and a third affinity reagent that binds specifically to the third type of unlabeled nucleotide; or (iv) a mixture of a second affinity reagent that binds specifically to the second type of nucleotide when the second type of nucleotide is unlabeled, and a third affinity reagent that binds specifically to the third type of unlabeled nucleotide;

wherein in the mixture described in (iii), the first affinity reagent comprises one or more first detectable labels, and the third affinity reagent comprises one or more third detectable labels; and

wherein in the mixture described in (iv), the second affinity reagent comprises one or more second detectable labels, and the third affinity reagent comprises one or more fourth detectable labels.

In any embodiments of the kits described herein, the kit may further comprise a fourth type of nucleotide, and wherein the fourth type of nucleotide is unlabeled (dark). In addition, any of the blue and violet dyes disclosed herein may be used as the first or the second label of the nucleotides or affinity reagents described in this section.

As one specific example, a kit may include the following nucleotides set: a dATP labeled with a blue dye A, a dTTP labeled with a violet dye B, a dCTP labeled with the blue dye A, a dCTP labeled with the violet dye B, and an unlabeled dGTP (dark G).

As another specific example, a kit may include the following nucleotides set: a dATP labeled with a blue dye A, a dTTP labeled with a violet dye B, a dCTP labeled with the blue dye C, a dCTP labeled with the violet dye D, and an unlabeled dGTP (dark G).

As another specific example, a kit may include the following nucleotides set: a dATP labeled with a blue dye A, a dTTP comprising a biotin moiety, a dCTP comprising a biotin moiety, a dCTP labeled with a blue dye A, and an unlabeled dGTP (dark G). Additionally, the kit may further include an affinity reagent comprising streptavidin labeled with one or more violet dye B, optionally through a cleavable linker.

As another specific example, a kit may include the following nucleotides set: a dATP labeled with a blue dye A, a dTTP comprising a first hapten that is a biotin moiety, a dCTP comprising a second hapten moiety, a dCTP labeled with a blue dye B, and an unlabeled dGTP (dark G). Additionally, the kit may further include a set of affinity reagents comprising streptavidin conjugated with one or more violet dye C, and a second hapten-binding partner conjugated with one or more violet dye D, each optionally through a cleavable linker.

In yet another example, a kit may include the following nucleotides set: a dATP comprising a chloroalkyl group (e.g., —(CH₂)₆Cl), a dTTP comprising a biotin moiety, a dCTP comprising a biotin moiety, a dCTP comprising a chloroalkyl group, and an unlabeled dGTP (dark G). The kit may further include a first affinity reagent comprising streptavidin labeled with one or more violet dyes, optionally through a cleavable linker. The kit may further include a second affinity reagent comprising HaloTag® labeled with one or more blue dyes, optionally through a cleavable linker.

In yet another example, a kit may include the following nucleotides set: a dATP comprising a first hapten comprising a chloroalkyl group (e.g., —(CH₂)₆Cl), a dTTP comprising a second hapten comprising a biotin moiety, a dCTP comprising a third hapten, and an unlabeled dGTP (dark G). The kit may further include a first affinity reagent comprising streptavidin labeled with one or more violet dye B, optionally through a cleavable linker. The kit may further include a second affinity reagent comprising HaloTag® labeled with one or more blue dye A, optionally through a cleavable linker. The kit may further include a third affinity reagent comprising a third hapten-binding partner and one or more blue dye C. The kit may further include a third affinity reagent comprising a third hapten-binding partner and one or more violet dye D.

In some embodiments of the kits described herein, the first excitation light source has a wavelength of about 350 nm to about 410 nm (e.g., about 405 nm), and the first emission filter has a detection wavelength of about 415 nm to about 450 nm. The second excitation light source has a wavelength of about 450 nm to about 460 nm (e.g., about 460 nm), and the second emission filter has a detection wavelength of about 480 nm to about 525 nm. In some other embodiments, the first excitation light source has a wavelength of about 450 nm to about 460 nm (e.g., about 460 nm), and the first emission filter has a detection wavelength of about 480 nm to about 525 nm. The second excitation light source has a wavelength of about 350 nm to about 410 nm (e.g., about 405 nm), and the second emission filter has a detection wavelength of about 415 nm to about 450 nm.

In addition to examples described above, the kit may comprise together at least one additional component. The further component(s) may be one or more of the components identified in a method set forth herein or in the Examples section below. Some non-limiting examples of components that can be combined into a kit of the present disclosure are set forth below. In some embodiments, the kit further comprises a DNA polymerase (such as a mutant DNA polymerase) and one or more buffer compositions. The kit may also include one or more antioxidants and/or ROS scavengers described herein. The antioxidants and/or ROS scavengers may be in a buffer solution or composition, which can be used to protect DNA (target polynucleotides and/or primer polynucleotides) and the dyes from photo damage during detection. Additional buffer composition may comprise a reagent can may be used to cleave the 3′ hydroxyl blocking group and/or the cleavable linker. For example, a water-soluble phosphines or water-soluble transition metal catalysts formed from a transition metal and at least partially water-soluble ligands, such as a palladium complex. Various components of the kit may be provided in a concentrated form to be diluted prior to use. In such embodiments a suitable dilution buffer may also be included. In further embodiments, the kit may include one or more solid supports. In some such embodiments, the solid support may comprise a plurality of oligonucleotides immobilized thereon. In some embodiments, the solid support comprises a flowcell, for example, a patterned flowcell comprising a plurality of nanowells.

In some embodiments of the kits described herein, the detectable labels (e.g., blue and violet dyes) may be covalently attached to a nucleotide via the nucleotide base. In some such embodiments, the labeled nucleotide may have the dye attached to the C5 position of a pyrimidine base or the C7 position of a 7-deaza purine base, optionally through a linker moiety. For example, the nucleobase may be 7-deaza adenine, and the dye is attached to the 7-deaza adenine at the C7 position, optionally through a linker. The nucleobase may be 7-deaza guanine, and the dye is attached to the 7-deaza guanine at the C7 position, optionally through a linker. The nucleobase may be cytosine, and the dye is attached to the cytosine at the C5 position, optionally through a linker. As another example, the nucleobase may be thymine or uracil and the dye is attached to the thymine or uracil at the C5 position, optionally through a linker. In any embodiments of the nucleotide or nucleotide conjugate described herein, the nucleotide or nucleotide conjugate may contain a 3′ hydroxyl blocking group. In other embodiments, when the nucleotide is unlabeled and a secondary labeling method is used, one or more blue dyes or violet dyes may be conjugated to an affinity reagent described herein, optionally through a cleavable linker. For example, one streptavidin may be labeled with two, three, four, five, or six molecules of the same violet dye to increase the fluorescent intensity of the incorporated nucleotide to be detected.

In any embodiments of the methods and kits described herein, when a label is described as excitable by a light source and detectable by an emission filter, it also refers to the nucleotide conjugated with such label (either direct labeling or secondary labeling through an affinity reagent) that is excitable by such light source and detectable by such emission filter.

3′ Hydroxyl Blocking Groups

In any embodiments of the methods and kits described herein, the nucleotides used in the incorporation mixture may have a blocking group covalently attached to the ribose or deoxyribose sugar of the nucleotide. In particular embodiments, the blocking group is at the 3′ OH position of the deoxyribose sugar of the nucleotide. Various 3′ OH blocking group are disclosed in WO 2004/018497 and WO 2014/139596, which are incorporated herein by reference. For example the blocking group may be azidomethyl (—CH₂N₃) or substituted azidomethyl (e.g., —CH(CHF₂)N₃ or CH(CH₂F)N₃), or allyl connecting to the 3′ oxygen atom of the ribose or deoxyribose moiety. In some embodiments, the 3′ blocking group is azidomethyl, forming 3′-OCH₂N₃ with the 3′ carbon of the ribose or deoxyribose.

In some other embodiments, the 3′ blocking group and the 3′ oxygen atoms form an acetal group of the structure

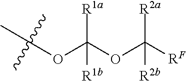

covalent attached to the 3′ carbon of the ribose or deoxyribose, wherein:

each R^{1a} and R^{1b} is independently H, C₁-C₆ alkyl, C₁-C₆ haloalkyl, C₁-C₆ alkoxy, C₁-C₆ haloalkoxy, cyano, halogen, optionally substituted phenyl, or optionally substituted aralkyl;

each R^{2a} and R^{2b} is independently H, C₁-C₆ alkyl, C₁-C₆ haloalkyl, cyano, or halogen;

alternatively, R^{1a} and R^{2a} together with the atoms to which they are attached form an optionally substituted five to eight membered heterocyclyl group;

R^{F} is H, optionally substituted C₂-C₆ alkenyl, optionally substituted C₃-C₇ cycloalkenyl, optionally substituted C₂-C₆ alkynyl, or optionally substituted (C₁-C₆ alkylene)Si(R^{3a})₃; and

each R^{3a} is independently H, C₁-C₆ alkyl, or optionally substituted C₆-C₁₀ aryl.

Additional 3′ OH blocking groups are disclosed in U.S. Publication No. 2020/0216891 A1, which is incorporated herein by reference in its entirety. Non-limiting examples of the acetal blocking group

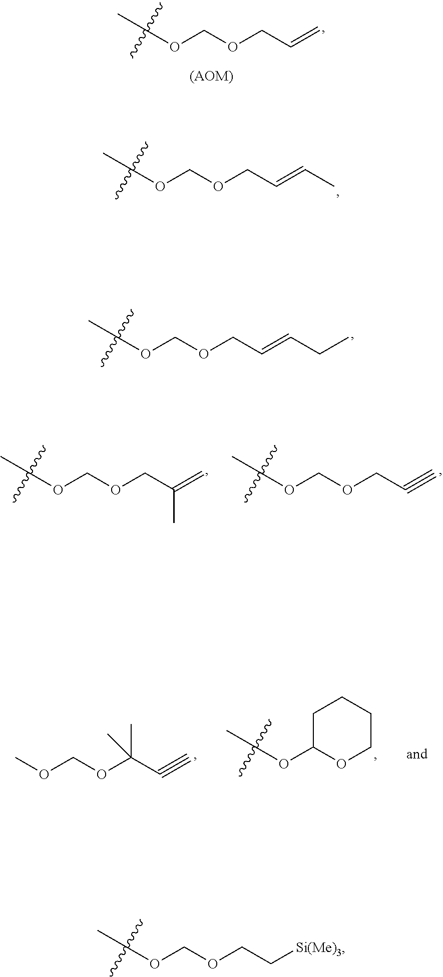

each covalently attached to the 3′ carbon of the ribose or deoxyribose.

Deprotection of the 3′ Hydroxyl Blocking Groups

In some embodiments, the azidomethyl 3′ hydroxy protecting group may be removed or deprotected by using a water-soluble phosphine reagent. Non-limiting examples include tris(hydroxymethyl)phosphine (THMP), tris(hydroxyethyl)phosphine (THEP) or tris(hydroxylpropyl)phosphine (THP or THPP). 3′-acetal blocking groups described herein may be removed or cleaved under various chemical conditions. For acetal blocking groups

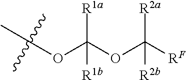

that contain a vinyl or alkenyl moiety, non-limiting cleaving condition includes a Pd(II) complex, such as Pd(OAc)₂ or allylPd(II) chloride dimer, in the presence of a phosphine ligand, for example tris(hydroxymethyl)phosphine (THMP), or tris(hydroxylpropyl)phosphine (THP or THPP). For those blocking groups containing an alkynyl group (e.g., an ethynyl), they may also be removed by a Pd(II) complex (e.g., Pd(OAc)₂ or allyl Pd(II) chloride dimer) in the presence of a phosphine ligand (e.g., THP or THMP).

Palladium Cleavage Reagents

In some embodiments, the 3′ hydroxyl blocking group described herein may be cleaved by a palladium catalyst. In some such embodiments, the Pd catalyst is water soluble. In some such embodiments, is a Pd(0) complex (e.g., Tris(3,3′,3″-phosphinidynetris(benzenesulfonato) palladium(0) nonasodium salt nonahydrate). In some instances, the Pd(0) complex may be generated in situ from reduction of a Pd(II) complex by reagents such as alkenes, alcohols, amines, phosphines, or metal hydrides. Suitable palladium sources include Na₂PdCl₄, Pd(CH₃CN)₂Cl₂, (PdCl(C₃H₅))₂, [Pd(C₃H₅)(THP)]Cl, [Pd(C₃H₅)(THP)₂]Cl, Pd(OAc)₂, Pd(Ph₃)₄, Pd(dba)₂, Pd(Acac)₂, PdCl₂(COD), and Pd(TFA)₂. In one such embodiment, the Pd(0) complex is generated in situ from Na₂PdCl₄. In another embodiment, the palladium source is allyl palladium(II) chloride dimer [(PdCl(C₃H₅))₂]. In some embodiments, the Pd(0) complex is generated in an aqueous solution by mixing a Pd(II) complex with a phosphine. Suitable phosphines include water soluble phosphines, such as tris(hydroxypropyl)phosphine (THP), tris(hydroxymethyl)phosphine (THMP), 1,3,5-triaza-7-phosphaadamantane (PTA), bis(p-sulfonatophenyl)phenylphosphine dihydrate potassium salt, tris(carboxyethyl)phosphine (TCEP), and triphenylphosphine-3,3′,3″-trisulfonic acid trisodium salt.

In some embodiments, the Pd(0) is prepared by mixing a Pd(II) complex [(PdCl(C₃H₅))₂] with THP in situ. The molar ratio of the Pd(II) complex and the THP may be about 1:2, 1:3, 1:4, 1:5, 1:6, 1:7, 1:8, 1:9, or 1:10. In some further embodiments, one or more reducing agents may be added, such as ascorbic acid or a salt thereof (e.g., sodium ascorbate). In some embodiments, the cleavage mixture may contain additional buffer reagents, such as a primary amine, a secondary amine, a tertiary amine, a carbonate salt, a phosphate salt, or a borate salt, or combinations thereof. In some further embodiments, the buffer reagent comprises ethanolamine (EA), tris(hydroxymethyl)aminomethane (Tris), glycine, sodium carbonate, sodium phosphate, sodium borate, 2-dimethylethanolamine (DMEA), 2-diethylethanolamine (DEEA), N,N,N′,N′-tetramethylethylenediamine(TEMED), or N,N,N′,N′-tetraethylethylenediamine (TEEDA), or combinations thereof. In one embodiment, the buffer reagent is DEEA. In another embodiment, the buffer reagent contains one or more inorganic salts such as a carbonate salt, a phosphate salt, or a borate salt, or combinations thereof. In one embodiment, the inorganic salt is a sodium salt.

Linkers

The fluorescent labels may be covalently attached to a nucleotide via a cleavable linker. Use of the term “cleavable linker” is not meant to imply that the whole linker is required to be removed. The cleavage site can be located at a position on the linker that ensures that part of the linker remains attached to the dye and/or substrate moiety after cleavage. Cleavable linkers may be, by way of non-limiting example, electrophilically cleavable linkers, nucleophilically cleavable linkers, photocleavable linkers, cleavable under reductive conditions (for example disulfide or azide containing linkers), oxidative conditions, cleavable via use of safety-catch linkers and cleavable by elimination mechanisms. The use of a cleavable linker to attach the dye compound to a substrate moiety ensures that the label can, if required, be removed after detection, avoiding any interfering signal in downstream steps.

Useful linker groups may be found in PCT Publication No. WO 2004/018493 (herein incorporated by reference), examples of which include linkers that may be cleaved using water-soluble phosphines or water-soluble transition metal catalysts formed from a transition metal and at least partially water-soluble ligands. In aqueous solution the latter form at least partially water-soluble transition metal complexes. Such cleavable linkers can be used to connect bases of nucleotides to labels such as the dyes set forth herein.

Particular linkers include those disclosed in PCT Publication No. WO 2004/018493 (herein incorporated by reference) such as those that include moieties of the formulae:

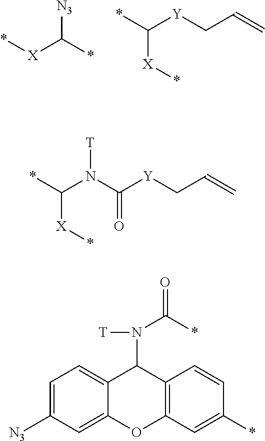

(wherein X is selected from the group comprising O, S, NH and NQ wherein Q is a C1-10 substituted or unsubstituted alkyl group, Y is selected from the group comprising O, S, NH and N(allyl), T is hydrogen or a C₁-C₁₀ substituted or unsubstituted alkyl group and * indicates where the moiety is connected to the remainder of the nucleotide). In some aspects, the linkers connect the bases of nucleotides to labels.

Additional examples of linkers include those disclosed in U.S. Publication No. 2016/0040225 (herein incorporated by reference), such as those include moieties of the formulae:

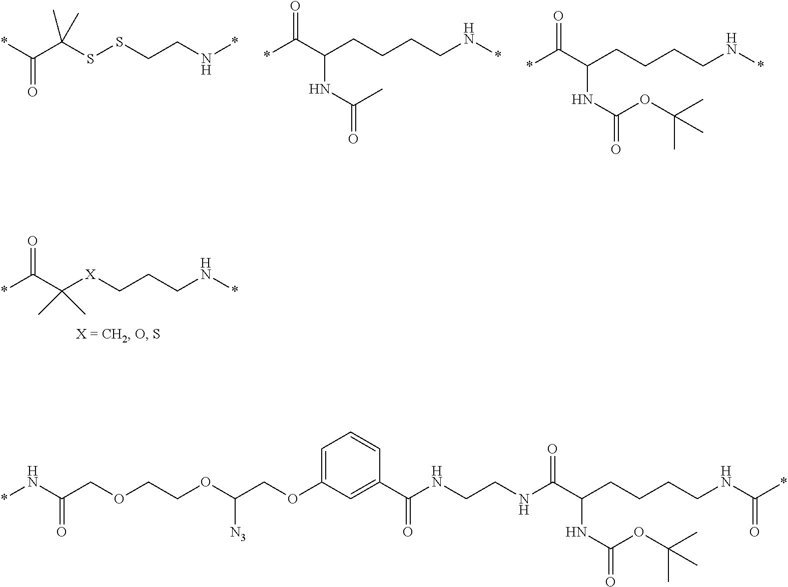

(wherein * indicates where the moiety is connected to the remainder of the nucleotide). The linker moieties illustrated herein may comprise the whole or partial linker structure between the nucleotides and the labels. The linker moieties illustrated herein may comprise the whole or partial linker structure between the nucleotides and the labels.

Additional examples of linkers include moieties of the formula:

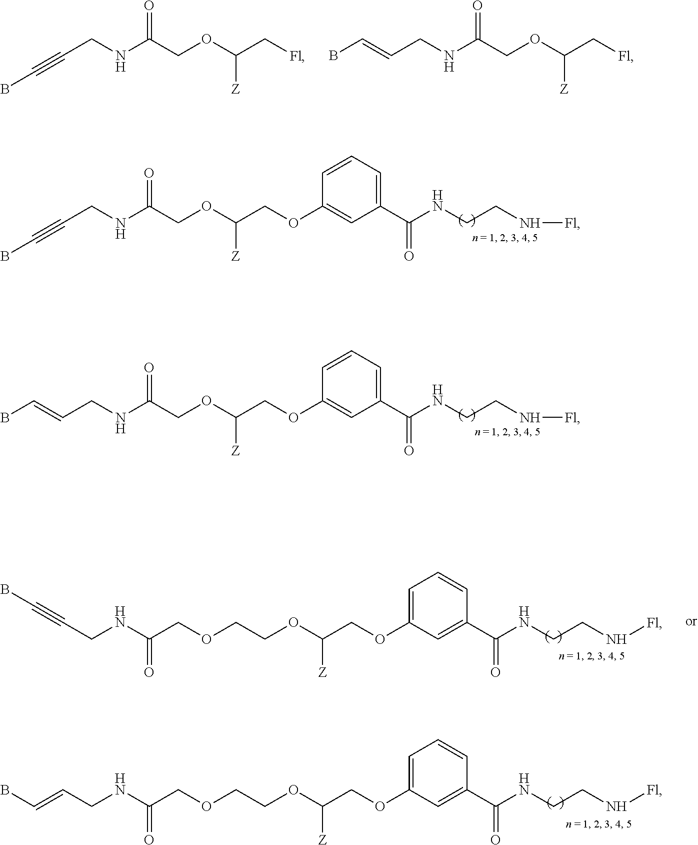

wherein B is a nucleobase; Z is —N₃ (azido), —O—C₁-C₆ alkyl, —O—C₂-C₆ alkenyl, or —O—C₂-C₆ alkynyl; and Fl comprises a dye moiety, which may contain additional linker structure. One of ordinary skill in the art understands that the dye compound described herein is covalently bounded to the linker by reacting a functional group of the dye compound (e.g., carboxyl) with a functional group of the linker (e.g., amino). In one embodiment, the cleavable linker comprises

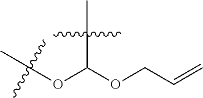 [SMILES: *OC(*)OCC=C]

(“AOL” linker moiety) where Z is —O-allyl.

A dye may be attached to any position on the nucleotide base, for example, through a linker. In particular embodiments, Watson-Crick base pairing can still be carried out for the resulting analog. Particular nucleobase labeling sites include the C5 position of a pyrimidine base or the C7 position of a 7-deaza purine base.

In some embodiments, when a nucleotide is unlabeled at the time of incorporation and relies on an affinity reagent to add detectable labels to the extended primer polynucleotide, the unlabeled nucleotide may still comprise a cleavable linker for attaching a hapten. The cleavable linker describe herein may also be used to attach the dyes to the affinity reagent when the secondary labeling method is used to add label(s) to the extended primer polynucleotide/target polynucleotide complex using an affinity reagent.

Labeled Nucleotides

Nucleotides labeled with the dyes described herein may have the formula:

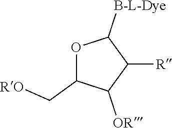

where Dye is a dye compound (label) moiety described herein (after covalent bonding between a functional group of the dye and a functional group of the linker “L”); B is a nucleobase, such as, for example uracil, thymine, cytosine, adenine, 7-deaza adenine, guanine, 7-deaza guanine, and the like; L is an optional linker which may or may not be present; R′ can be H, or —OR′ is monophosphate, diphosphate, triphosphate, thiophosphate, a phosphate ester analog, —O— attached to a reactive phosphorous containing group, or —O— protected by a blocking group; R″ is H or OH; and R′″ is H, a 3′ OH blocking group described herein, or —OR′″ forms a phosphoramidite. Where —OR′″ is phosphoramidite, R′ is an acid-cleavable hydroxyl protecting group which allows subsequent monomer coupling under automated synthesis conditions. In some further embodiments, B comprises

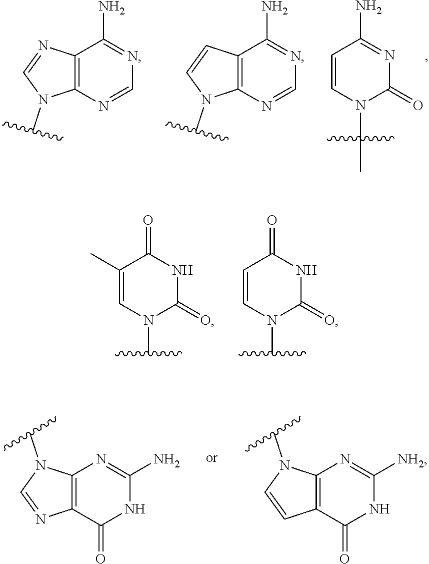

or optionally substituted derivatives and analogs thereof. In some further embodiments, the labeled nucleobase comprises the structure

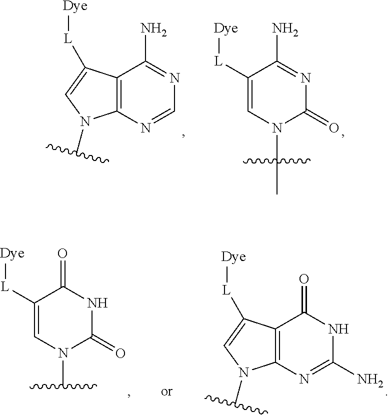

In a particular embodiment, the blocking group is separate and independent of the dye compound, i.e., not attached to it. Alternatively, the dye may comprise all or part of the 3′ OH blocking group. Thus R′″ can be a 3′ OH blocking group which may or may not comprise the dye compound.

In yet another alternative embodiment, there is no blocking group on the 3′ carbon of the pentose sugar and the dye (or dye and linker construct) attached to the base, for example, can be of a size or structure sufficient to act as a block to the incorporation of a further nucleotide. Thus, the block can be due to steric hindrance or can be due to a combination of size, charge and structure, whether or not the dye is attached to the 3′ position of the sugar.

In still yet another alternative embodiment, the blocking group is present on the 2′ or 4′ carbon of the pentose sugar and can be of a size or structure sufficient to act as a block to the incorporation of a further nucleotide.

In some embodiments, the linker (between dye and nucleotide) and blocking group are both present and are separate moieties. In particular embodiments, the linker and blocking group are both cleavable under the same or substantially similar conditions. Thus, deprotection and deblocking processes may be more efficient because only a single treatment will be required to remove both the dye compound and the blocking group. However, in some embodiments a linker and blocking group need not be cleavable under similar conditions, instead being individually cleavable under distinct conditions.

The disclosure also encompasses polynucleotides incorporating dye compounds. Such polynucleotides may be DNA or RNA comprised respectively of deoxyribonucleotides or ribonucleotides joined in phosphodiester linkage. Polynucleotides may comprise naturally occurring nucleotides, non-naturally occurring (or modified) nucleotides other than the labeled nucleotides described herein or any combination thereof, in combination with at least one modified nucleotide (e.g., labeled with a dye compound) as set forth herein. Polynucleotides according to the disclosure may also include non-natural backbone linkages and/or non-nucleotide chemical modifications. Chimeric structures comprised of mixtures of ribonucleotides and deoxyribonucleotides comprising at least one labeled nucleotide are also contemplated.

Non-limiting exemplary labeled nucleotide conjugates as described herein include:

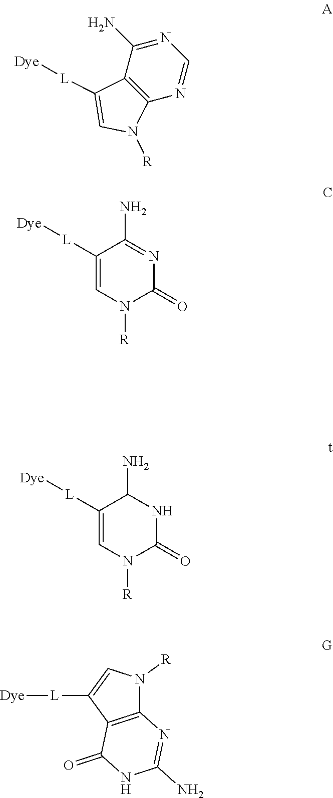

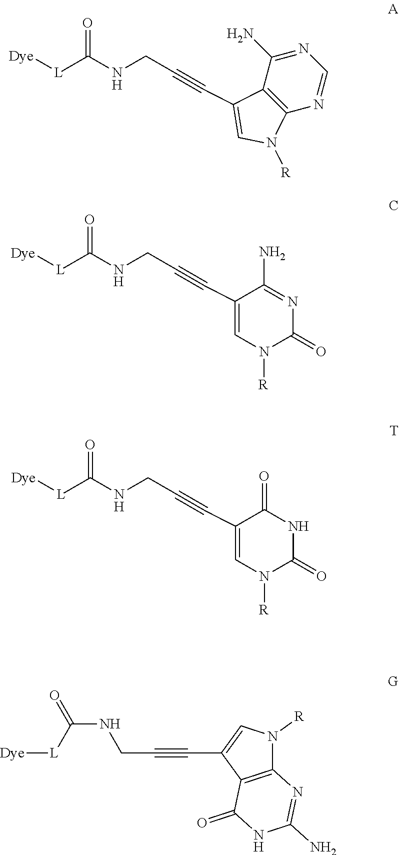

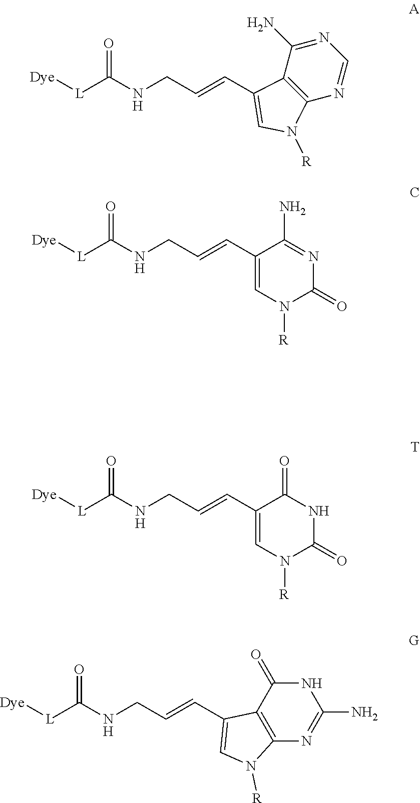

wherein L represents a linker and R represents a ribose or deoxyribose moiety as described above, or a ribose or deoxyribose moiety with the 5′ position substituted with mono-, di- or tri-phosphates.

In some embodiments, non-limiting exemplary fully functionalized nucleotide conjugates including a cleavable linker and a fluorescent moiety are shown below:

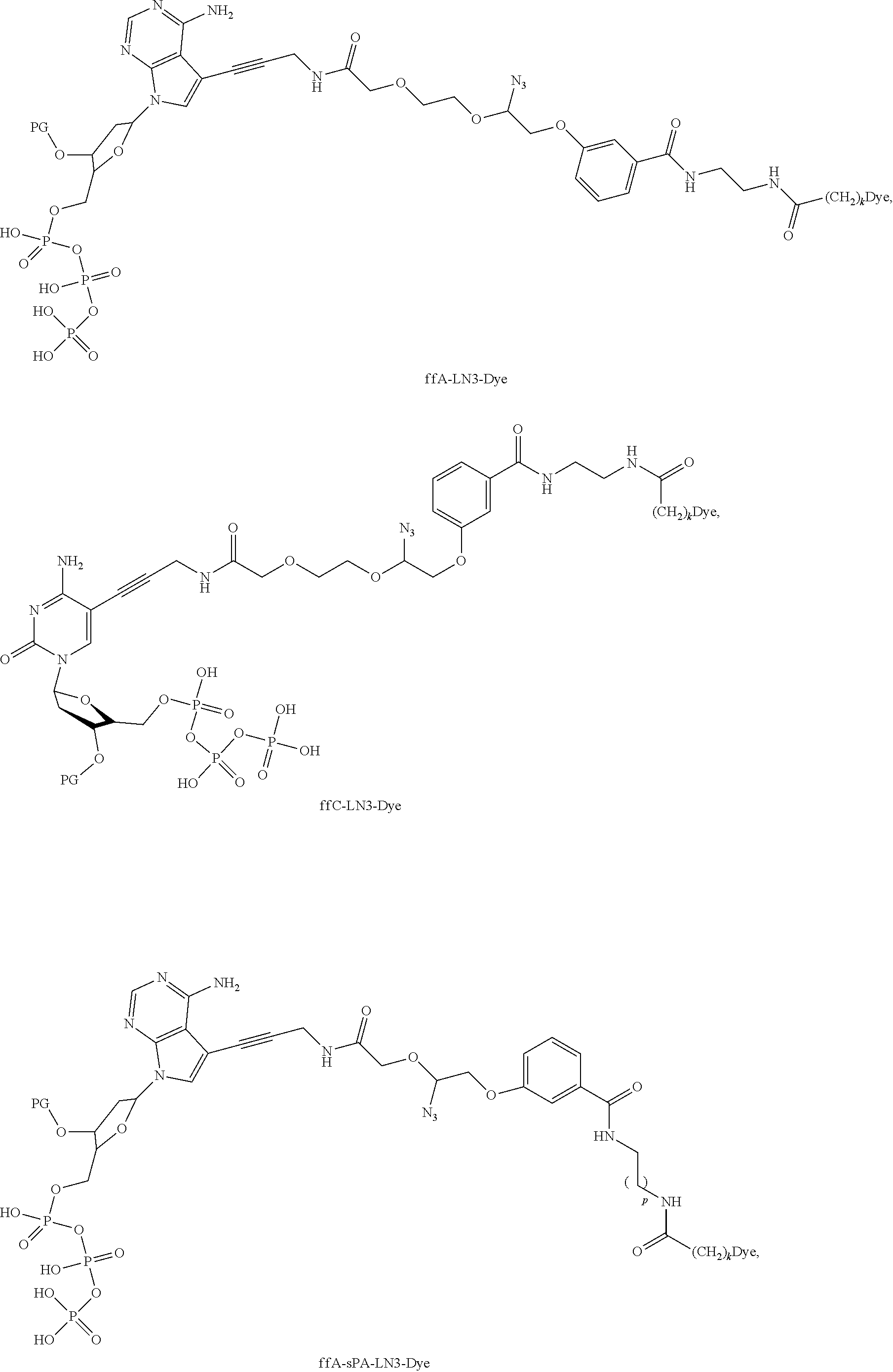

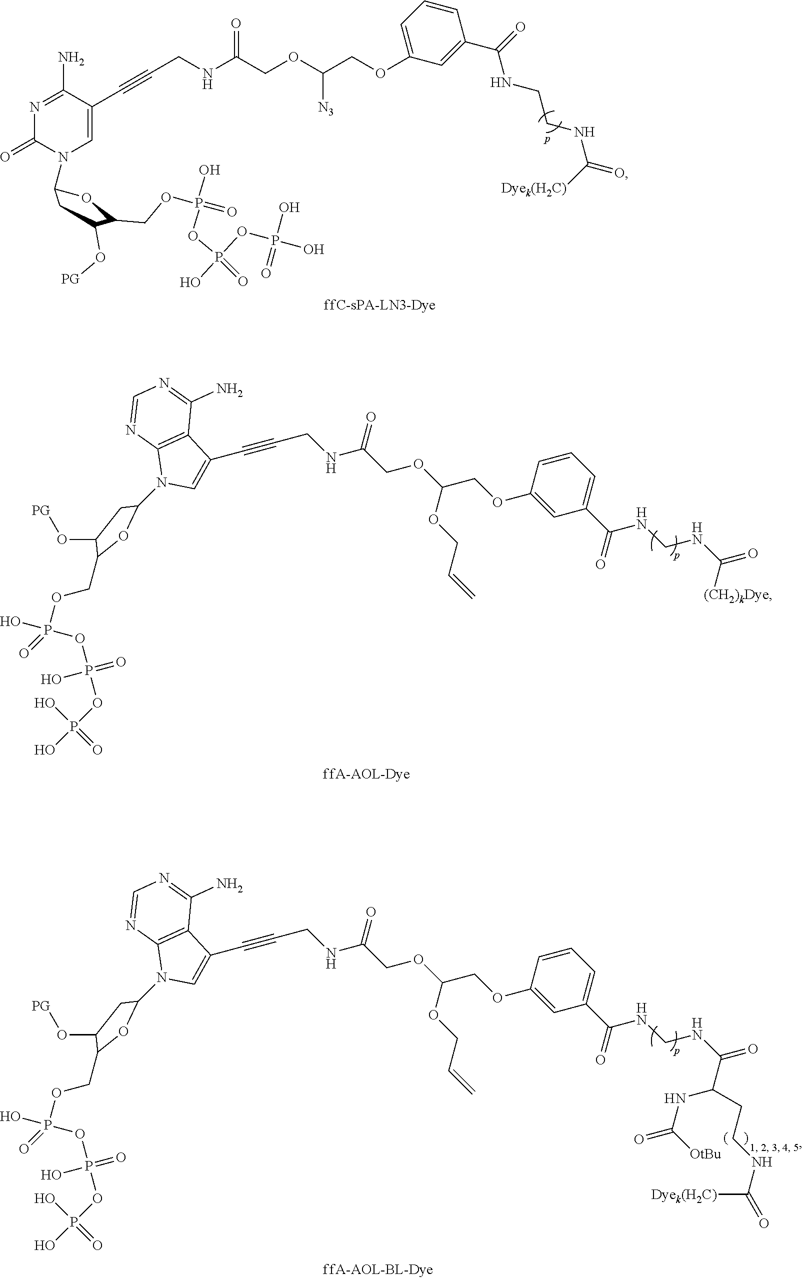

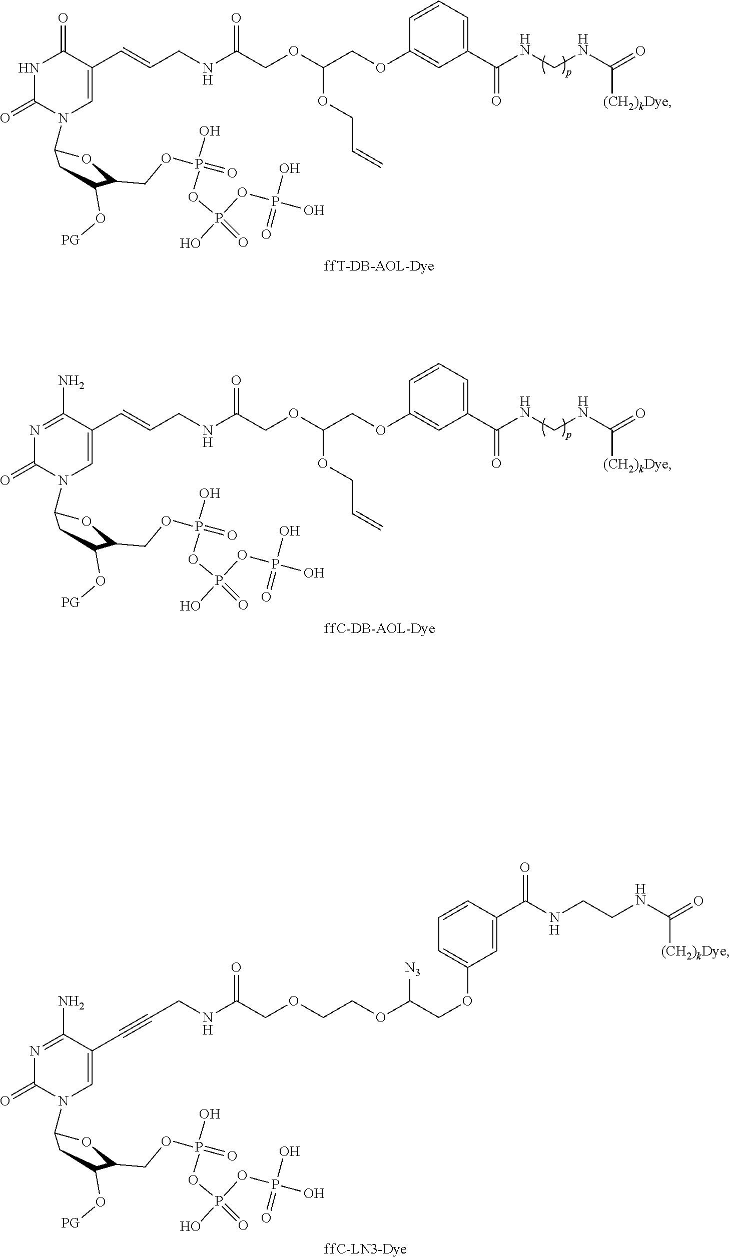

wherein PG stands for the 3′ OH blocking groups described herein; p is an integer of 1, 2, 3, 4, 5, 6, 7, 8, 9, or 10; and k is 0, 1, 2, 3, 4, or 5. In one embodiment, —O-PG is AOM. In another embodiment, —O-PG is —O-azidomethyl. In one embodiment, k is 5. In some further embodiments, p is 1, 2 or 3; and k is 5.

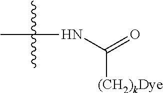

refers to the connection point of the Dye with the cleavable linker as a result of a reaction between an amino group of the linker moiety and the carboxyl group of the Dye (i.e., a blue dye or a violet dye described herein). In any embodiments of the labeled nucleotide described herein, the nucleotide is a nucleotide triphosphate. Alternatively, when the ffN is not labeled, the Dye moiety may be replaced with a functional moiety (e.g., a hapten) that can enable the binding of the unlabeled nucleotide with an affinity reagent described herein.

### EXAMPLES

Additional embodiments are disclosed in further detail in the following examples, which are not in any way intended to limit the scope of the claims.

### Example 1. Assessment of DNA Photodamage Using Violet Irradiation at 405 nm

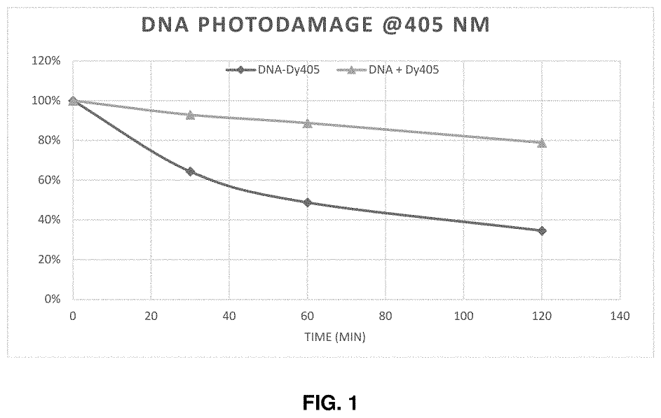

In this example, the DNA damaged caused by a violet light at 405 nm was assessed. A single strand DNA in a Tris buffer (pH=8, 100 mM) was irradiated under a violet LED for 2 hours, either covalently attached to a violet dye DY405, or in the presence of DY405 in the buffer. It was observed that when DY405 was covalently attached to the 5′ terminal of DNA, the photodamage to the DNA caused by the irradiation was substantially increased as compared to when the DNA was mixed in the buffer solution with DY405. The result was illustrated in FIG. 1.

### Example 2. Sequencing by Synthesis Using Blue/Violet Two-Channel MiSeq® System

In this example, sequencing by synthesis was performed on a MiSeq® instrument configured as a 2-channel Blue/Violet. Standard sequencing reagents were used. The incorporation mixture for standard SBS is summarized in Table 1. The sequenced library presented in those data is PhiX.

For the standard SBS incorporation mix, the violet dye used is DY405. Both ffT and ffC were labeled with DY405. Blue Dye A was used for labeling ffA and ffC. Green ffNs were also introduced to reduce signal intensity in the violet and blue channels in order to get a square scatter plot with preferred shape for post-analysis. ffG was unlabeled (“dark G”). The structures of nucleotides in the incorporation mixture are illustrated below. Both ffC-sPA-DY405 and ffT-LN3-DY405 were prepared by using standard ffN coupling reaction by reacting pppC-sPA-NH₂ or pppT-LN3-NH₂ with Dy405-NHS (5 mg).

TABLE 1
Standard SBS Incorporation mix composition

| ffNs | Nucleotide Label |
|---|---|
| ffG | Dark (unlabeled) |
| ffC | Blue dye A (coumarin dye) |
| ffC | DY405 |
| ffT | DY405 |
| ffT | NR550S0 (green dye) |
| ffA | Blue dye A (coumarin dye) |
| ffA | NR550S0 (green dye) |

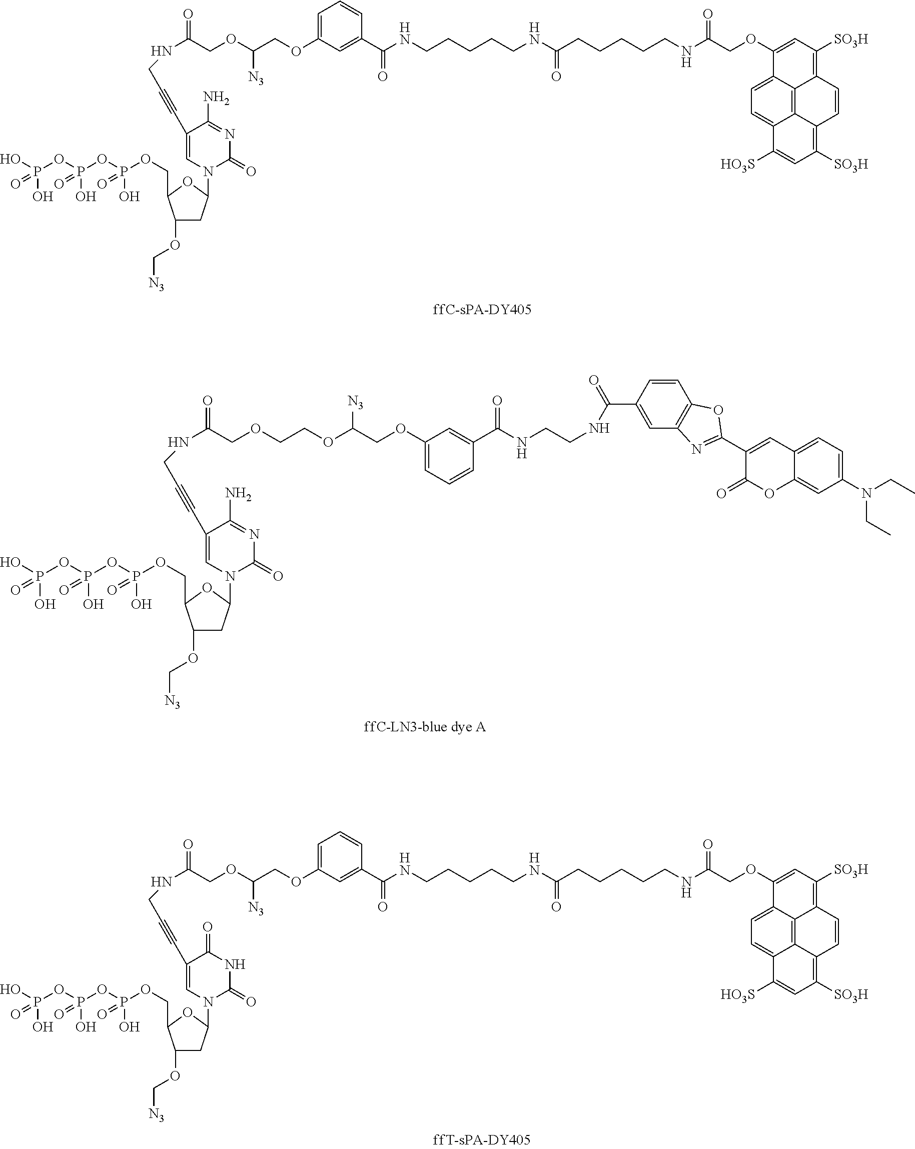

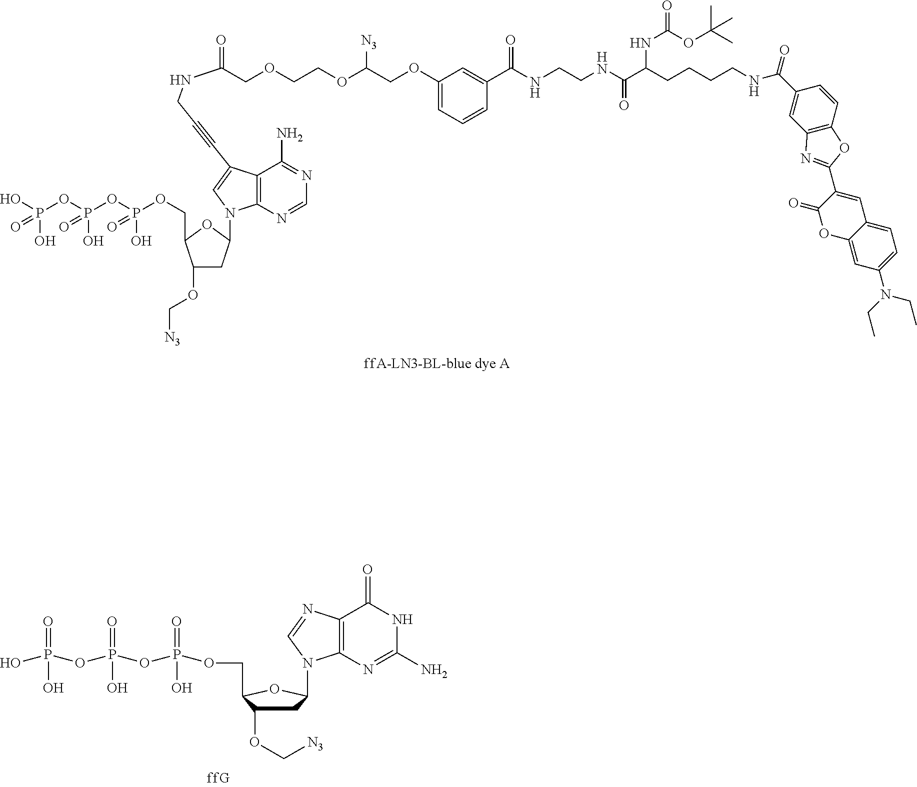

Preparation of DY405-labeled streptavidin. First, DY405 was converted to DY405-NHS by reacting DY405 with N,N,N′,N′-tetramethyl-O—(N-succinimidyl)uronium tetrafluoroborate) (1.5 eq.) in the presence of Hunig's base and TSTU in anhydrous DMA for 30 minutes. Second, streptavidin powder was dissolved in water and NaHCO₃ buffer. The DY405-NHS prepared from the first step was transferred into the streptavidin solution and incubated at room temperature for 1 hour with occasional mixture. Then 5M NaCl solution was added to the reaction mixture. The reaction product was purified by removing excess dye using a Thermo Fisher Dye removal column. Quantification of the reaction product showed the final dye/protein ratio was about 3.1 to 3.4.

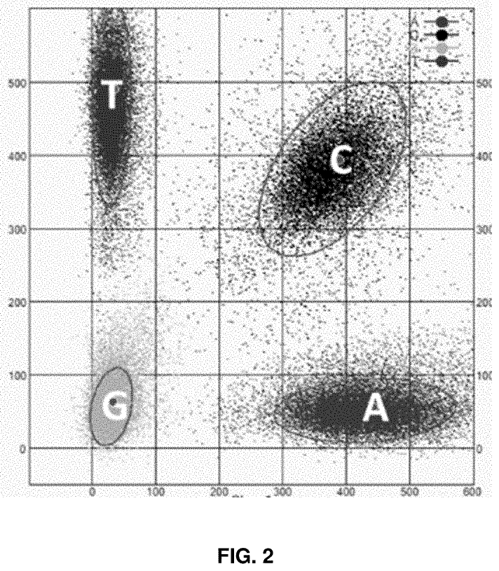

For the secondary labeling SBS, secondary labeling was used for dTTP and dCTP. In the secondary labeling SBS incorporation mixture, ffT was unlabeled and comprised a biotin moiety. ffC was both unlabeled and labeled with a blue dye A, and ffA was labeled with a blue dye A. The incorporation mixture is summarized in Table 2. In the secondary labeling SBS, an extra step was required in the sequencing recipe after the standard incorporation. After incorporation of one nucleotide, a solution of DY405-labeled streptavidin was flushed on the flowcell and incubated for 25 s at 60° C., followed by a buffer wash before performing the first and second imaging events. The Streptavidin-DY405 solution contained: 5 ug/ml of Streptavidin-DY405, NaCl, EDTA, Tween® 20 (polysorbate 20) in 5 mM Tris, pH 7.5. Commercial MiSeq® flowcell was used in this experiment. FIG. 2 illustrates a scatter plot obtained with the secondary labeling SBS, demonstrating the usability of this sequencing method.

TABLE 2
Secondary labeling SBS incorporation mix composition

| ffNs | Nucleotide Label/Hapten |
|---|---|
| ffG | Dark |
| ffC | Blue dye A (coumarin dye) |
| ffC | Biotin |
| ffT | Biotin |
| ffT | NR550S0 (green dye) |
| ffA | Blue dye A (coumarin dye) |
| ffA | NR550S0 (green dye) |

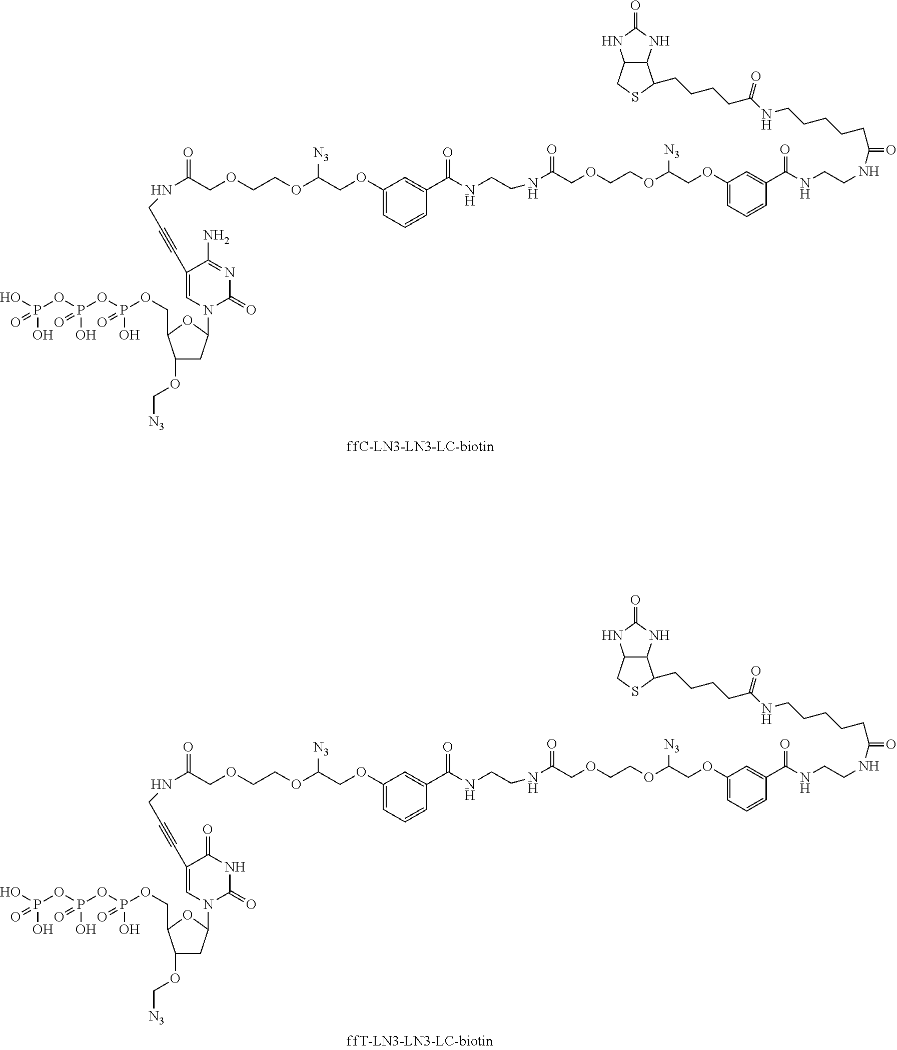 [SMILES: [N-]=[N+]=NCOC1CC(n2cc(C#CCNC(=O)COCCOC(COc3cccc(C(=O)NCCNC(=O)COCCOC(COc4cccc(C(=O)NCCNC(=O)CCCCCNC(=O)CCCCC5SCC6NC(=O)NC65)c4)N=[N+]=[N-])c3)N=[N+]=[N-])c(N)nc2=O)OC1COP(=O)(O)OP(=O)(O)OP(=O)(O)O ; [N-]=[N+]=NCOC1CC(n2cc(C#CCNC(=O)COCCOC(COc3cccc(C(=O)NCCNC(=O)COCCOC(COc4cccc(C(=O)NCCNC(=O)CCCCCNC(=O)CCCCC5SCC6NC(=O)NC65)c4)N=[N+]=[N-])c3)N=[N+]=[N-])c(=O)[nH]c2=O)OC1COP(=O)(O)OP(=O)(O)OP(=O)(O)O]

TABLE 3
Primary sequencing metrics for both SBS conditions (50 cycles per read).

| | % Phasing | % Prephasing | Aligned (%) | Error Rate (%) |
|---|---|---|---|---|
| R1 | 0.497 | 0.237 | 95.27 | 0.67 ± 0.02 |
| R2 | 0.498 | 0.212 | 44.24 | 15.40 ± 0.13 |

Table 3 shows primary sequencing metrics for both the secondary labeling SBS and the standard SBS (R1=secondary labeling SBS and R2=standard SBS). The results show that blue/violet two-channel sequencing is compatible with the modified method involving secondary labeling of the violet dye.

However, the % Phasing and the % Signal decay observed for a 50-cycle run using blue/violet (B/V) channels are much higher than for a standard blue/green (B/G) sequencing (Table 4).

TABLE 4
Primary metrics for B/V and B/G sequencing (50 cycles).

| | | | Aligned | Error Rate | % Signal decay |
|---|---|---|---|---|---|
| SBS | % Phasing | % Prephasing | (%) | (%) | (% Intensity left) |
| B/V | 0.497 | 0.237 | 95.27 | 0.67 | 71 |
| B/G | 0.098 | 0.125 | — | 0.13 | 87 |

Further experiments were conducted to understand the causation of the signal decay in connection with the nucleotide incorporation time and violet illumination time. It was discovered that both increased dose of violet light and increase in violet light exposure time exacerbated signal decay. However, by increasing the nucleotide incorporation time, % phasing was substantially decreased and as a result, signal decay was also improved. Furthermore, signal decay was also improved by using a brighter flowcell with decreased loss of violet light and shorter violet exposure time (e.g., reducing violet exposure time from 250 ms to 170 ms).

1. A method for determining the sequence of a target polynucleotide, comprising:
(a) contacting a primer polynucleotide with a mixture comprising one or more of a first type of nucleotide, a second type of nucleotide, a third type of nucleotide, and a fourth type of nucleotide, wherein the primer polynucleotide is complementary to at least a portion of the target polynucleotide;
(b) incorporating one type of nucleotide from the mixture to the primer polynucleotide to produce an extended primer polynucleotide;
(c) performing a first imaging event using a first excitation light source and collecting a first emission signal from the extended primer polynucleotide with a first emission filer; and
(d) performing a second imaging event using a second excitation light source and collecting a second emission signal from the extended primer polynucleotide with a second emission filter;
wherein one of the first excitation light source and the second excitation light source has a wavelength of about 350 nm to about 410 nm, and the other one of the first excitation light source and the second excitation light source has a wavelength of about 450 nm to about 460 nm; and
wherein one of the first emission filter and the second emission filter has a detection wavelength of about 415 nm to about 450 nm, and the other one of the first emission filter and the second emission filter has a detection wavelength of about 480 nm to about 525 nm.

2. The method of claim 1, wherein the first type of nucleotide is labeled with a first detectable label that is excitable by the first excitation light source and detectable by the first emission filter.

3. The method of claim 1, wherein the second type of nucleotide is labeled with a second detectable label that is excitable by the second excitation light source and detectable by the second emission filter, and wherein the second type of detectable label is spectrally distinguishable from the first type of detectable label.

4. The method of claim 1, wherein the third type of nucleotide is labeled both with a first detectable label and a second detectable label, and the third type of nucleotide is excitable by both the first excitation light source and the second excitation light source.

5. The method of claim 1, wherein the third type of nucleotide comprises a mixture of a third type of nucleotide labeled with a third label and a third type of nucleotide labeled with a fourth label, wherein the third label is excitable by the first excitation light source and detectable by the first emission filter, and wherein the fourth label is excitable by the second excitation light source and detectable by the second emission filter.

6. The method of claim 1, wherein each of the first type, the second type and the third type of nucleotide is unlabeled, and the method further comprising: contacting the extended primer polynucleotide with a set of affinity reagents prior to the first imaging event, wherein at least one affinity reagent in the set binds specifically to the incorporated first type, second type, or third type of nucleotide.

7. The method of claim 6, wherein the set of affinity reagents comprises: a first affinity reagent that binds specifically to the first type of nucleotide, a second affinity reagent that binds specifically to the second type of nucleotide, and wherein the first affinity reagent comprises one or more first detectable labels that are excitable by the first excitation light source and detectable by the first emission filter, the second affinity reagent comprises one or more second detectable labels that are excitable by the second excitation light source and detectable by the second emission filter, and wherein the first detectable label is spectrally distinguishable from the second detectable label.

8. (canceled)

9. The method of claim 7, wherein both the first affinity reagent and the second affinity reagent bind specifically to the third type of nucleotide.

10. The method of claim 7, wherein the set of affinity reagents further comprises a third affinity reagent that binds specifically to the third type of nucleotide, and wherein the third affinity reagent comprises one or more third detectable labels that are excitable by the first excitation light source and detectable by the first emission filter, and one or more fourth detectable labels that are excitable by the second excitation light source and detectable by the second emission filter.

11. The method of claim 7, wherein the first type of nucleotide comprises a first hapten, and the first affinity reagent comprises a first hapten-binding partner that specifically binds to the first hapten.

12. The method of claim 11, wherein the first hapten comprises a biotin moiety and the first hapten-binding partner comprises streptavidin.

13. The method of claim 7, wherein the second type of nucleotide comprises a second hapten, and the second affinity reagent comprises a second hapten-binding partner that specifically binds to the second hapten.

14. The method of claim 13, wherein the second hapten comprises a chloroalkyl group and the second hapten-binding partner comprises HaloTag®.

15. The method of claim 1, wherein the first type of nucleotide is labeled with a first detectable label, the second type of nucleotide is unlabeled, the third type of nucleotide is both unlabeled and labeled with the first detectable label, and the first detectable label is excitable by the first excitation light source and detectable by the first emission filter, and the method further comprising: contacting the extended primer polynucleotide with an affinity reagent prior to the first imaging event, wherein the affinity reagent binds specifically to the second type of unlabeled nucleotide or the third type of unlabeled nucleotide, and wherein the affinity reagent comprises one or more second detectable labels that are excitable by the second excitation light source and detectable by the second emission filter.

16. (canceled)

17. The method of claim 15, wherein the affinity reagent comprises streptavidin, and both the second type of nucleotide and the third type unlabeled nucleotide comprise a biotin moiety.

18. The method of claim 1, wherein the first type of nucleotide is unlabeled, the second type of nucleotide is labeled with a second detectable label, the third type of nucleotide is both unlabeled and labeled with the second detectable label, and the second detectable label is excitable by the second excitation light source and detectable by the second emission filter, and the method further comprising: contacting the extended primer polynucleotide with an affinity reagent prior to the first imaging event, wherein the affinity reagent binds specifically to the first type of unlabeled nucleotide or the third type of unlabeled nucleotide, and the affinity reagent comprises one or more first detectable labels that are excitable by the first excitation light source and detectable by the first emission filter.

19. (canceled)

20. The method of claim 18, wherein the affinity reagent comprises streptavidin, and both the first type of nucleotide and the third type unlabeled nucleotide comprise a biotin moiety.

21. The method of claim 1, wherein the fourth type of nucleotide is unlabeled (dark), or is labeled with a fluorescent moiety that has no emission from either the first imaging event or the second imaging event.

22. The method of claim 1, wherein the four types of nucleotides comprise dATP, dCTP, dGTP and dTTP or dUTP, or non-natural nucleotide analogs thereof, and wherein each of the four types of nucleotides in the mixture has a 3′ hydroxyl blocking group.

23. (canceled)

24. The method of claim 22, further comprising: (e) removing the 3′ hydroxyl blocking group from the incorporated nucleotide after the second imaging event, and prior to the next sequencing cycle.

25. The method of claim 24, further comprising:
repeating steps (a)-(e) for multiple cycles; and
determining the sequence of the target polynucleotide based on the sequentially incorporated nucleotides.

26. The method of claim 25, wherein steps (a)-(e) are repeated for at least 50 cycles.

27.-32. (canceled)

33. The method of claim 1, wherein the target polynucleotide is immobilized to a solid support, and the solid support comprises a plurality of immobilized target polynucleotides, and the method is carried out in an array format by sequencing the plurality of immobilized target polynucleotides in parallel.

34. (canceled)

35. The method of claim 33, wherein the solid support comprises a patterned flow cell, comprising the plurality of immobilized target polynucleotides inside the nanowells of the patterned flow cell.

36. (canceled)

37. The method of claim 33, wherein the density of the immobilized target polynucleotides on the solid support is from about 100 k/mm² to about 300 k/mm².

38. A kit for sequencing application, comprising:
a first type of nucleotide labeled with a first detectable label;
a second type of nucleotide labeled with a second detectable label;
a third type of nucleotide labeled with the first detectable label; and
a third type of nucleotide labeled with the second detectable label;
wherein the first detectable label and the second detectable label are spectrally distinguishable from one another, the first detectable label is excitable by a first light source and detectable by a first emission filter, and the second detectable label is excitable by a second light source and detectable by a second emission filter;
wherein one of the first excitation light source and the second excitation light source has a wavelength of about 350 nm to about 410 nm, and the other one of the first excitation light source and the second excitation light source has a wavelength of about 450 nm to about 460 nm; and
wherein one of the first emission filter and the second emission filter has a detection wavelength of about 415 nm to about 450 nm, and the other one of the first emission filter and the second emission filter has a detection wavelength of about 480 nm to about 525 nm.

39. A kit for sequencing application, comprising:
a first type of nucleotide labeled with a first detectable label;
a second type of nucleotide labeled with a second detectable label;
a third type of nucleotide labeled with a third detectable label; and
a third type of nucleotide labeled with a fourth detectable label;
wherein the first detectable label and the second detectable label are spectrally distinguishable from one another, the first detectable label is excitable by a first light source and detectable by a first emission filter, and the second detectable label is excitable by a second light source and detectable by a second emission filter;
wherein the third detectable label and the fourth detectable label are spectrally distinguishable from one another, the third detectable label is excitable by the first light source and detectable by the first emission filter, and the fourth detectable label is excitable by the second light source and detectable by the second emission filter;
wherein one of the first excitation light source and the second excitation light source has a wavelength of about 350 nm to about 410 nm, and the other one of the first excitation light source and the second excitation light source has a wavelength of about 450 nm to about 460 nm; and
wherein one of the first emission filter and the second emission filter has a detection wavelength of about 415 nm to about 450 nm, and the other one of the first emission filter and the second emission filter has a detection wavelength of about 480 nm to about 525 nm.

40. A kit for sequencing application, comprising:
a first type of unlabeled nucleotide;
a second type of unlabeled nucleotide;
a third type of unlabeled nucleotide; and
a set of affinity reagents comprising:
a first affinity reagent that binds specifically to the first type of unlabeled nucleotide; and
a second affinity reagent that binds specifically to the second type of unlabeled nucleotide;
wherein the first affinity reagent comprises one or more first detectable labels that are excitable by a first excitation light source and detectable by a first emission filter, the second affinity reagent comprises one or more second detectable labels that are excitable by a second excitation light source and detectable by a second emission filter, and wherein the first detectable label is spectrally distinguishable from the second detectable label;
wherein one of the first excitation light source and the second excitation light source has a wavelength of about 350 nm to about 410 nm, and the other one of the first excitation light source and the second excitation light source has a wavelength of about 450 nm to about 460 nm; and
wherein one of the first emission filter and the second emission filter has a detection wavelength of about 415 nm to about 450 nm, and the other one of the first emission filter and the second emission filter has a detection wavelength of about 480 nm to about 525 nm.

41.-46. (canceled)

47. A kit for sequencing application, comprising:
a first type of nucleotide either unlabeled or labeled with a first detectable label;
a second type of nucleotide either unlabeled or labeled with a second detectable label, wherein one of the first type of nucleotide and the second type of nucleotide is unlabeled;
a third type of unlabeled nucleotide, and a third type of nucleotide labeled with the same detectable label as either the first or the second type of nucleotide, wherein the first detectable label and the second detectable label are spectrally distinguishable from one another, the first detectable label is excitable by a first light source and detectable by a first emission filter, and the second detectable label is excitable by a second light source and detectable by a second emission filter; and
an affinity reagent comprising either a first affinity reagent that binds specifically to the third type of unlabeled nucleotide and the first type of nucleotide if the first type of nucleotide is unlabeled, or a second affinity reagent that binds specifically to the third type of unlabeled nucleotide and the second type of nucleotide if the second type of nucleotide is unlabeled, wherein the first affinity reagent comprises one or more first detectable labels and the second affinity reagent comprises one or more second detectable labels;
wherein one of the first excitation light source and the second excitation light source has a wavelength of about 350 nm to about 410 nm, and the other one of the first excitation light source and the second excitation light source has a wavelength of about 450 nm to about 460 nm; and
wherein one of the first emission filter and the second emission filter has a detection wavelength of about 415 nm to about 450 nm, and the other one of the first emission filter and the second emission filter has a detection wavelength of about 480 nm to about 525 nm.

48.-59. (canceled)
